(19) United States
(12) Patent Application Publication
(10) Pub. No.: US 2024/0216352 A1
(43) Pub. Date: Jul. 4, 2024

(54) LPA1 ANTAGONISTS FOR TREATING INTERSTITIAL LUNG DISEASE

(71) Applicant: Bristol-Myers Squibb Company, Princeton, NJ (US)

(72) Inventors: Giridhar S. TIRUCHERAI, Kendall Park, NJ (US); Edgar D. CHARLES, Westfield, NJ (US); Aryeh FISCHER, Denver, CO (US); Shiwei TAO, Hillsborough, NJ (US)

(21) Appl. No.: 18/394,005

(22) Filed: Dec. 22, 2023

Related U.S. Application Data
(60) Provisional application No. 63/519,692, filed on Aug. 15, 2023.
(60) Provisional application No. 63/476,992, filed on Dec. 23, 2022.

(51) Int. Cl. A61K 31/4439; A61K 31/4418; A61K 31/496; A61P 11/00

(52) U.S. Cl. CPC A61K 31/4439; A61K 31/4418; A61K 31/496; A61P 11/00

(57) ABSTRACT

This disclosure relates to methods of treating interstitial lung disease by administering (1S,3S)-3-((2-methyl-6-(1-methyl-5-(((methyl(propyl)carbamoyl)oxy)methyl)-1H-1,2,3-triazol-4-yl)pyridin-3-yl)oxy)cyclohexane-1-carboxylic acid (a LPA₁ antagonist).

### CROSS-REFERENCE TO RELATED APPLICATIONS

This application claims the priority benefit of U.S. Provisional Application No. 63/476,992, filed Dec. 23, 2022, and U.S. Provisional Application No. 63/519,692, filed Aug. 15, 2023, which are each incorporated by reference herein in their entireties.

### TECHNICAL FIELD

This disclosure relates to methods of treating interstitial lung disease by administering (1S,3S)-3-((2-methyl-6-(1-methyl-5-(((methyl(propyl)carbamoyl)-oxy)methyl)-1H-1,2,3-triazol-4-yl)pyridin-3-yl)oxy)cyclohexane-1-carboxylic acid (an LPA₁ antagonist).

### BACKGROUND

The interstitial lung diseases (ILDs) are a group of heterogeneous lung disorders classified together based on shared clinical features, parenchymal lung scarring (fibrosis) and/or inflammation with varying patterns of lung injury by imaging or histopathology. ILD may arise due to identifiable causes such as an underlying systemic autoimmune disease (e.g., systemic sclerosis or rheumatoid arthritis), environmental exposure (e.g., asbestos or silica), or result from medication toxicity, but is often idiopathic in nature. Idiopathic pulmonary fibrosis (IPF), one of the more common and most devastating types of ILD, is a chronic, progressive, and typically fatal lung disease of unknown cause characterized by worsening dyspnea, cough, loss of lung function due to scar formation in the lung and must have the pathological and radiographic pattern known as usual interstitial pneumonia (UIP) (Meltzer et al., Orphanet J. Rare Dis. 2008, 3:8). Beyond IPF, some patients with other forms of ILD develop a progressive fibrotic phenotype, characterized by worsening respiratory symptoms, lung function, progressive fibrosis on imaging and early mortality.

To date, two approved treatments, pirfenidone and nintedanib, significantly reduce the decline in lung function in patients with IPF and both appear to have modest effects on progression-free survival (Noble et al., Lancet 2011, 377 (9779), 1760−1769; King et al., N. Engl. J. Med. 2014, 370 (22), 2083−2092; Richeldi et al., New Engl J Med 2014, 370 (22), 2071−2082). However, many patients progress despite treatment. Another treatment option is lung transplantation, which has been shown to improve mortality in carefully selected patients, but not without complications (Kistler et al., BMC Pulmonary Med. 2014; 14:139). Despite these advances, there remains a large unmet need for a safe, well-tolerated and effective therapy for IPF that improves pulmonary function, delays disease progression, and reduces mortality.

Because of the clinical and pathophysiological similarities among IPF and other forms of progressive pulmonary fibrosis (PPF), it has been suggested that such disorders have a common pathobiologic mechanism regardless of the cause, with resultant progressive lung fibrosis, and thus could have a similar response to treatment as IPF (Raghu et al., Am. J. Respir. Crit. Care Med. 2022, 205, e18-e47; du Bois et al., Am. J. Respir. Crit. Care Med. 2012, 186, 712−715; Flaherty et al., N. Engl. J. Med. 2019, 381 (18), 1718−1727). Indeed, in the recently published INBUILD trial, patients with PPF with diverse etiologies were treated with nintedanib or placebo. Those treated with nintedanib had slower progression of lung fibrosis than those who received placebo, as demonstrated by a lower annual rate of decline in forced vital capacity (FVC) over the 52-week study period (Flaherty et al., N. Engl. J. Med. 2019, 381 (18), 1718−1727). The absolute treatment effects in the PPF study were similar in magnitude to those observed in the pivotal INPULSIS trials that led to approval of nintedanib for treatment of IPF. Furthermore, the IMPULSIS and INBUILD trials demonstrated that IPF and PPF patients were similar with respect to their rate of FVC decline. Based on data from the INBUILD trial, health authorities have also approved nintedanib for patients with PPF. Nonetheless, as many patients are unable to tolerate nintedanib due to gastrointestinal side effects, and because some patients may progress despite ongoing treatment with nintedanib, there remains an unmet need for well-tolerated, effective therapies for PPF (Flaherty et al., N. Engl. J. Med. 2019, 381 (18), 1718−1727).

Overall, there is a high unmet need for effective and tolerable treatments for patients with IPF and non-IPF, PPF that exhibit disease progression. Fibrotic diseases such as these can be mediated by LPA, which signals via six LPA receptors (LPA₁-6). Signalling via LPA₁ appears to be fundamental in the pathogenesis of fibrotic diseases.

U.S. Patent Application Publication No. 2017/0360759 (PCT Application Publication No. WO2017/223016) discloses certain antagonists of lysophosphatidic acid (LPA) receptors for use in treating LPA-dependent or LPA-mediated conditions or diseases such as fibrosis of various organs, including the lung.

The compound of (1S,3S)-3-((2-methyl-6-(1-methyl-5-(((methyl(propyl)carbamoyl)-oxy)methyl)-1H-1,2,3-triazol-4-yl)pyridin-3-yl)oxy)cyclohexane-1-carboxylic acid (hereafter referred as to “Compound A”) is described in U.S. Patent Application Publication No. 2017/0360759.

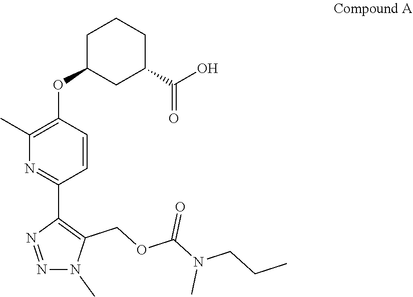 [SMILES: CCCN(C)C(=O)OCc1c(-c2ccc(O[C@H]3CCC[C@H](C(=O)O)C3)c(C)n2)nnn1C]

Compound A is a potent LPA₁ antagonist in vitro (LPA₁K_{b}=6.9 nM in CHO cells overexpressing human LPA₁ and LPA₁K_{b}=5.9 nM in normal human lung fibroblasts). Compound A is currently in clinical development as a therapy for IPF and PF-ILD.

The present disclosure provides methods of treating interstitial lung disease using Compound A.

### BRIEF DESCRIPTION OF THE DRAWINGS

FIG. 1 shows a powder X-ray diffraction (PXRD) pattern of Form A.

FIG. 2 shows a differential scanning calorimetry (DSC) thermogram of Form A.

FIG. 3 shows a thermogravimetric analysis (TGA) thermogram of Form A.

FIG. 4 shows moisture sorption isotherms of Form A.

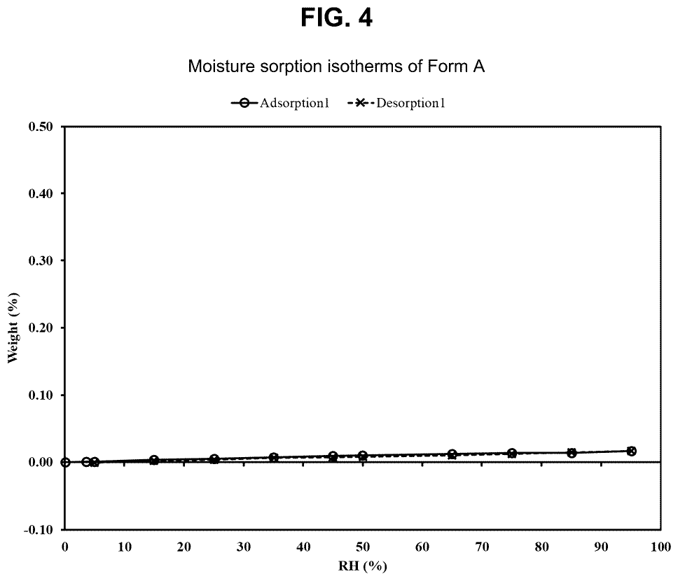

FIG. 5 shows an outline of the Phase 2 trial.

FIG. 6 shows the rate of change in ppFVC in patients treated with placebo or Compound A.

FIG. 7 shows the change in FVC (mL) in patients treated with placebo or Compound A.

FIG. 8 shows the absolute change from baseline in FVC (mL) in patients treated with placebo or Compound A.

FIG. 9 shows the rate of change in ppFVC according to background antifibrotic use in patients treated with placebo or Compound A.

FIG. 10 shows the change from baseline in ppFVC percentage (primary estimand) in PPF subjects treated with placebo or Compound A.

FIG. 11 shows the change from baseline in FVC (mL) (primary estimand) in PPF subjects treated with placebo or Compound A.

FIG. 12 shows the change from baseline in ppFVC percentage (primary estimand) of PPF subjects with UIP treated with placebo or Compound A.

FIG. 13 shows the change from baseline in FVC (mL) (primary estimand) in PPF subjects without UIP treated with placebo or Compound A.

FIG. 14 shows the rate of change in ppFVC of PPF subjects with and without UIP treated with placebo or Compound A.

FIG. 15 shows the change from baseline in ppFVC percentage (primary estimand) of PPF subjects taking antifibrotics that were treated with placebo and Compound A.

FIG. 16 shows the change from baseline in FVC (mL) (primary estimand) in PPF subjects not taking antifibrotics that were treated with placebo or Compound A.

### SUMMARY

In some aspects, the present disclosure provides a method of treating interstitial lung disease, the method comprising administering to a subject in need thereof about 120 mg/day of Compound A:

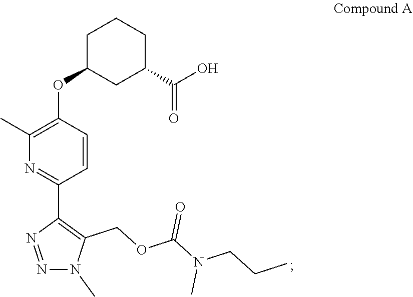 [SMILES: CCCN(C)C(=O)OCc1c(-c2ccc(O[C@H]3CCC[C@H](C(=O)O)C3)c(C)n2)nnn1C]

or an equivalent amount of a pharmaceutically acceptable salt thereof. In some aspects, Compound A, or the pharmaceutically acceptable salt thereof, is administered once daily.

In some aspects, Compound A, or the pharmaceutically acceptable salt thereof, is administered twice daily. In some aspects, about 60 mg of Compound A, or an equivalent amount of a pharmaceutically acceptable salt thereof, is administered twice daily. In some aspects, Compound A, or the pharmaceutically acceptable salt thereof, is administered orally. In some aspects, Compound A, or the pharmaceutically acceptable salt thereof, is administered as a tablet.

In some aspects, the subject being treated is concomitantly being treated with one or more therapies for interstitial lung disease. In some aspects, the one or more therapies is pirfenidone. In some aspects, the one or more therapies is ninedanib.

In some aspects, Compound A, or the pharmaceutically acceptable salt thereof, is administered to the subject with food. In some aspects, Compound A, or the pharmaceutically acceptable salt thereof, is administered to the subject without food.

In some aspects, the interstitial lung disease is idiopathic pulmonary fibrosis (IPF). In some aspects, the interstitial lung disease is progressive pulmonary fibrosis (PPF).

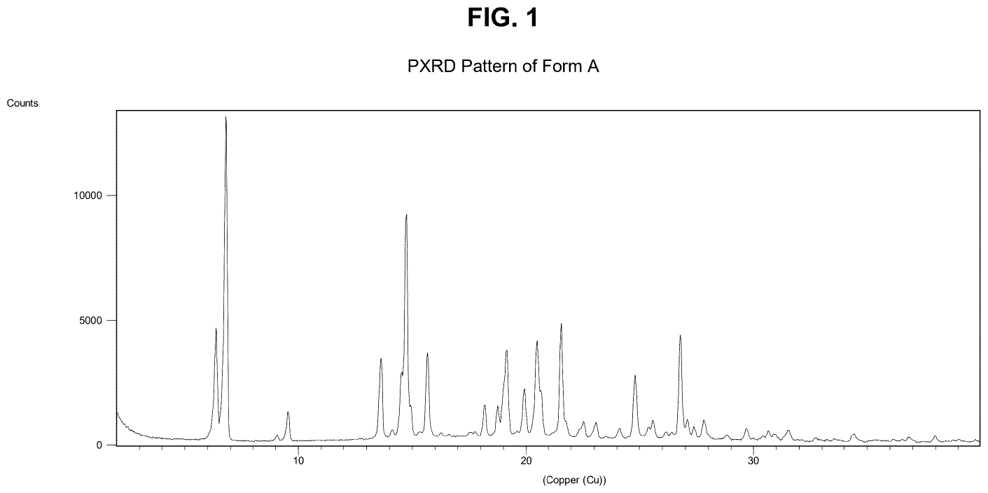

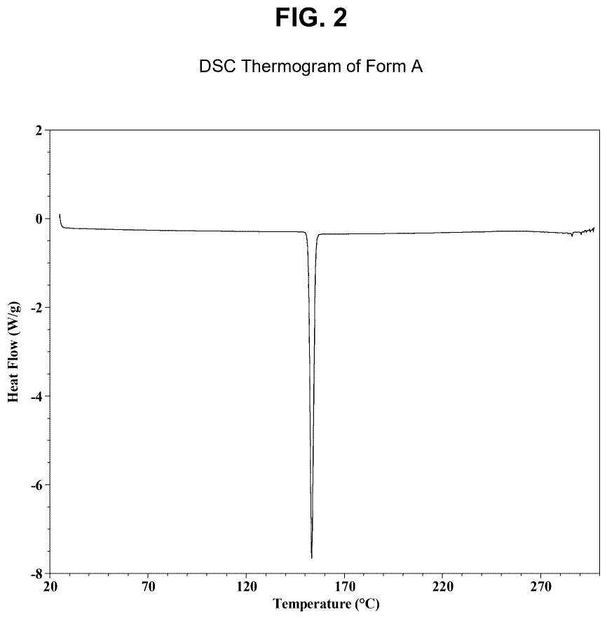

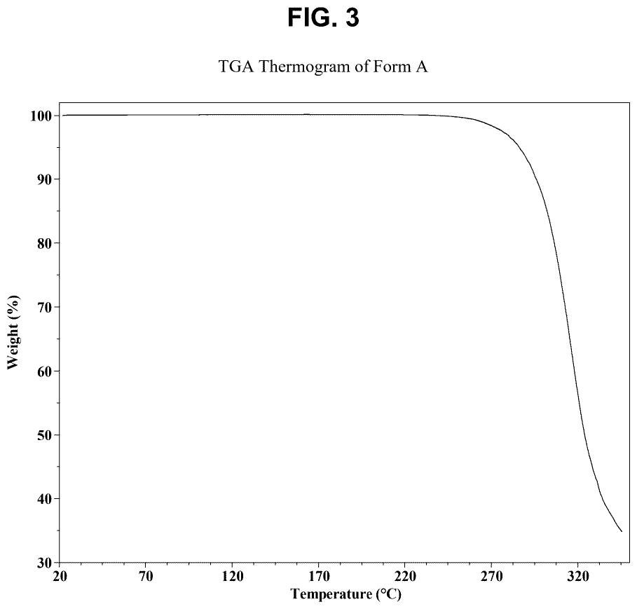

In some aspects, Compound A comprises the crystal form characterized by at least one of the following:
a) single crystal structure having unit cell parameters substantially equal to

| Crystal system, space group | Triclinic, P1 | |
|---|---|---|
| Unit cell dimensions | a = 6.53 ± | alpha = |
| | 0.10 \acute{Å} | 92.8 ± 1.0° |
| | b = 13.06 ± | beta = |
| | 0.10 \acute{Å} | 95.5 ± 1.0° |
| | c = 14.04 ± | gamma = |
| | 0.10 \acute{Å} | 93.0 ± 1.0° |

| | |
|---|---|
| Volume | 1189(20) \acute{Å}³ |
| Density (calculated) | 1.239 g/cm³ |
| Temperature | room temperature |

wherein measurement of the single crystal structure is at room temperature;
b) a powder x-ray diffraction pattern substantially the same as shown in FIG. 1;
c) a powder x-ray diffraction pattern comprising 2 or more peaks at 2θ values selected from 6.4±0.2, 6.8±0.2, 9.6±0.2, 13.6±0.2, 15.7±0.2, 18.2±0.2, 19.9±0.2, 21.6±0.2, 24.8±0.2 and 26.8±0.2 (obtained at room temperature and CuKαλ=1.5418 Å);
d) a powder x-ray diffraction pattern comprising 3 or more peaks at 2θ values selected from 6.4±0.2, 6.8±0.2, 9.6±0.2, 13.6±0.2, 14.1±0.2, 14.5±0.2, 14.7±0.2, 15.7±0.2, 18.2±0.2, 18.7±0.2, 19.2±0.2, 19.9±0.2, 20.5±0.2, 21.6±0.2, 22.5±0.2, 23.1±0.2, 24.1±0.2, 24.8±0.2, 25.6±0.2, 26.8±0.2, 27.1±0.2 and 27.8±0.2 (obtained at room temperature and CuKαλ=1.5418 Å);
e) a differential scanning calorimetry thermogram substantially similar to the one as shown in FIG. 2;
f) a differential scanning calorimetry thermogram with an endotherm having an onset at about 152° C.; and/or
g) a thermal gravimetric analysis thermogram substantially similar to the one as shown in FIG. 3.

In some aspects, the subject experiences a smaller decline in forced vital capacity (FVC) after a treatment period compared to an untreated subject. In some aspects, the subject experiences a greater time to first disease progression event after a treatment period than an untreated subject, wherein the first disease progression event is selected from:
absolute percentage predicted forced vital capacity (ppFVC) of ≥10% from baseline;
acute exacerbation of lung fibrosis;
respiratory hospitalization
lung transplantation; and
all-cause mortality.

In some aspects, the subject experiences a greater time to first disease progression event after a treatment period than an untreated subject, wherein the first disease progression event is selected from:
absolute predicted forced vital capacity (ppFVC) decline of ≥10% from baseline;
acute exacerbation of lung fibrosis;
lung fibrosis-related hospitalization; and
all-cause mortality.

In some aspects, the subject experiences a smaller increase in cough domain score as measured by the Living with Pulmonary Fibrosis (L-PF) questionnaire over a treatment period than an untreated subject. In some aspects, the subject experiences a smaller increase in dyspnea score as measured by the Living with Pulmonary Fibrosis (L-PF) questionnaire over a treatment period than an untreated subject.

In some aspects, the present disclosure provides the use of about 120 mg/day of Compound A:

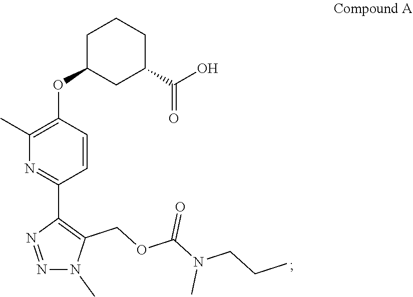 [SMILES: CCCN(C)C(=O)OCc1c(-c2ccc(O[C@H]3CCC[C@H](C(=O)O)C3)c(C)n2)nnn1C]

or an equivalent amount of a pharmaceutically acceptable salt thereof, for treating interstitial lung disease.

In some aspects, the present disclosure provides the use of about 120 mg/day of Compound A:

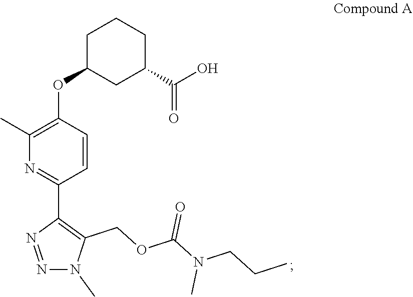 [SMILES: CCCN(C)C(=O)OCc1c(-c2ccc(O[C@H]3CCC[C@H](C(=O)O)C3)c(C)n2)nnn1C]

or an equivalent amount of a pharmaceutically acceptable salt thereof, in the manufacture of a medicament for treating interstitial lung disease. In some aspects, the interstitial lung disease is idiopathic pulmonary fibrosis (IPF). In some aspects, the interstitial lung disease is progressive pulmonary fibrosis (PPF).

### DETAILED DESCRIPTION

The present disclosure provides a method of treating interstitial lung disease, the method comprising administering to a subject in need thereof about 120 mg/day of Compound A:

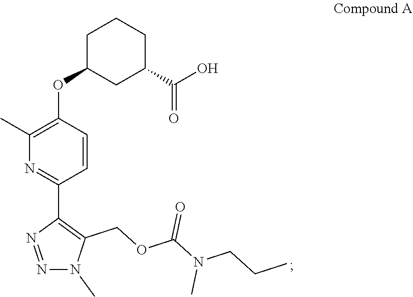 [SMILES: CCCN(C)C(=O)OCc1c(-c2ccc(O[C@H]3CCC[C@H](C(=O)O)C3)c(C)n2)nnn1C]

or an equivalent amount of a pharmaceutically acceptable salt thereof.

In order that the present description can be more readily understood, certain terms are first defined. Additional definitions are set forth throughout the detailed description.

### I. Definitions

Unless otherwise stated, the following terms used in this application, including the specification and claims, have the definitions given below. It must be noted that, as used in the specification and the appended claims, the singular forms “a,” “an,” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. Furthermore, use of the term “including” as well as other forms, such as “include,” “includes,” and “included,” is not limiting. The section headings used herein are for organizational purposes only and are not to be construed as limiting the subject matter described.

All measurements are subject to experimental error and are within the spirit of the invention.

The term “about” as used in connection with a numerical value throughout the specification and the claims denotes an interval of accuracy, familiar and acceptable to a person skilled in the art. Such interval of accuracy is ±10%.

The term “acceptable” with respect to a formulation, composition or ingredient, as used herein, means having no persistent detrimental effect on the general health of the subject being treated.

The terms “administration of” and or “administering a” compound or composition should be understood to mean providing a compound or composition described herein to one or more subjects.

As used herein, “amorphous” refers to a solid form of a molecule, atom, and/or ions that is not crystalline. An amorphous solid does not display a definitive X-ray diffraction pattern.

The term “antagonist”, as used herein, refers to a molecule such as a compound, which diminishes, inhibits, or prevents the action of another molecule or the activity of a receptor site. Antagonists include, but are not limited to, competitive antagonists, non competitive antagonists, uncompetitive antagonists, partial agonists and inverse agonists.

As used herein, the term “BP” refers to blood pressure, the term “SBP” refers to systolic blood pressure and the term “DBP” refers to diastolic blood pressure.

The terms “co-administration” or the like, as used herein, are meant to encompass administration of the selected therapeutic agents to a single subject, and are intended to include treatment regimens in which the agents are administered by the same or different route of administration or at the same or different time.

As used herein, the term “DSC” refers to differential scanning calorimetry. The term “TGA” refers to thermogravimetric analysis.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case can be determined using techniques, such as a dose escalation study.

As used herein, the phrase “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, “polymorphs” refer to crystalline forms having the same chemical structure but different spatial arrangements of the molecules and/or ions forming the crystals.

The term “room temperature” generally means approximately 22° C., but can vary up or down by 7° C.

The terms “subject” and “participant” are used interchangeably and encompass mammals. Examples of mammals include, but are not limited to, humans, chimpanzees, apes, monkey, cattle, horses, sheep, goats, swine, rabbits, dogs, cats, rodents, rats, mice guinea pigs, and the like. In one aspect, the mammal is a human.

As used herein, “substantially pure,” when used in reference to a crystal form, means a compound having a purity greater than 90 weight %, including greater than 90, 91, 92, 93, 94, 95, 96, 97, 98, and 99 weight %, and also including equal to about 100 weight % of the crystal form of Compound A, based on the weight of the compound. The remaining material comprises other form(s) of the compound, and/or reaction impurities and/or processing impurities arising from its preparation. For example, a crystal form of Compound A can be deemed substantially pure in that it has a purity greater than 90 weight %, as measured by means that are at this time known and generally accepted in the art, where the remaining less than 10 weight % of material comprises other form(s) of Compound A and/or reaction impurities and/or processing impurities.

When the term “substantially in accordance” is used in relation to PXRD, or XRPD patterns, it is to be understood that measurement of the peak locations for a given crystalline form of the same compound will vary within a margin of error. It is also to be understood that the intensities of the peaks can vary between different PXRD scans of the same crystalline form of the same compound. The relative intensities of the different peaks are not meant to be limiting to a comparison of different PXRD scans.

The terms “treat,” “treating,” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### II. Compound A

Compound A is described in U.S. Patent Application Publication No. 2017/0360759.

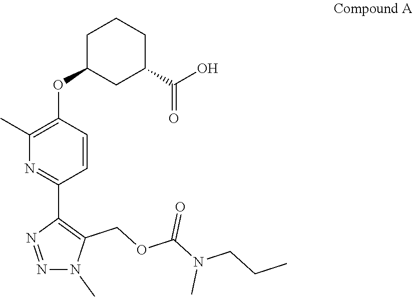 [SMILES: CCCN(C)C(=O)OCc1c(-c2ccc(O[C@H]3CCC[C@H](C(=O)O)C3)c(C)n2)nnn1C]

In some aspects, Compound A comprises crystalline form “Form A.” When dissolved, a crystalline form of Compound A loses its crystalline structure, and is therefore referred to as a solution of Compound A. All forms of the present invention, however, can be used for the preparation of liquid formulations in which the drug is dissolved or suspended. In addition, the crystalline Form A of Compound A can be incorporated into solid formulations.

As used herein, a PXRD (powder x-ray diffraction) or XRPD (x-ray powder diffraction) pattern “comprising” or having a number of peaks selected from a specified group of peaks, is intended to include PXRD patterns having additional peaks that are not included in the specified group of peaks. For example, a PXRD pattern comprising four or more peaks, preferably five or more, at 2θ values selected from: A, B, C, D, E, F, G, and H, is intended to include a PXRD pattern having: (a) four or more peaks, preferably five or more, at 2θ values selected from: A, B, C, D, E, F, G, and H; and (b) zero or more peaks that are not one of peaks A, B, C, D, E, F, G, and H.

In some aspects, Form A is characterized by a single crystal structure having unit cell parameters substantially equal to

| Crystal system, space group | Triclinic, P1 | |
|---|---|---|
| Unit cell dimensions | a = 6.53 ± | alpha = |
| | 0.10 \acute{Å} | 92.8 ± 1.0° |
| | b = 13.06 ± | beta = |
| | 0.10 \acute{Å} | 95.5 ± 1.0° |
| | c = 14.04 ± | gamma = |
| | 0.10 \acute{Å} | 93.0 ± 1.0° |

| | |
|---|---|
| Volume | 1189(20) \acute{Å}³ |
| Density (calculated) | 3 1.239 g/cm³ |
| Temperature | room temperature |

wherein measurement of the single crystal structure is at room temperature.

In some aspects, Form A has a a powder x-ray diffraction pattern substantially the same as shown in FIG. 1.

In some aspects, Form A has a powder x-ray diffraction pattern comprising 2 or more peaks at 2θ values selected from 6.4±0.2, 6.8±0.2, 9.6±0.2, 13.6±0.2, 15.7±0.2, 18.2±0.2, 19.9±0.2, 21.6±0.2, 24.8±0.2 and 26.8±0.2 (obtained at room temperature and CuKαλ=1.5418 Å). In some aspects, Form A has a powder x-ray diffraction comprising 3 or more peaks at 2θ values selected from 6.4±0.2, 6.8±0.2, 9.6±0.2, 13.6±0.2, 15.7±0.2, 18.2±0.2, 19.9±0.2, 21.6±0.2, 24.8±0.2 and 26.8±0.2 (obtained at room temperature and CuKαλ=1.5418 Å). In some aspects, Form A has a powder x-ray diffraction comprising 4 or more peaks at 2θ values selected from 6.4±0.2, 6.8±0.2, 9.6±0.2, 13.6±0.2, 15.7±0.2, 18.2±0.2, 19.9±0.2, 21.6±0.2, 24.8±0.2 and 26.8±0.2 (obtained at room temperature and CuKαλ=1.5418 Å).

In some aspects, Form A has a powder x-ray diffraction pattern comprising 3 or more peaks at 2θ values selected from 6.4±0.2, 6.8±0.2, 9.6±0.2, 13.6±0.2, 14.1±0.2, 14.5±0.2, 14.7±0.2, 15.7±0.2, 18.2±0.2, 18.7±0.2, 19.2±0.2, 19.9±0.2, 20.5±0.2, 21.6±0.2, 22.5±0.2, 23.1±0.2, 24.1±0.2, 24.8±0.2, 25.6±0.2, 26.8±0.2, 27.1±0.2 and 27.8±0.2 (obtained at room temperature and CuKαλ=1.5418 Å). In some aspects, Form A has a powder x-ray diffraction pattern comprising 4 or more peaks at 2θ values selected from 6.4±0.2, 6.8±0.2, 9.6±0.2, 13.6±0.2, 14.1±0.2, 14.5±0.2, 14.7±0.2, 15.7±0.2, 18.2±0.2, 18.7±0.2, 19.2±0.2, 19.9±0.2, 20.5±0.2, 21.6±0.2, 22.5±0.2, 23.1±0.2, 24.1±0.2, 24.8±0.2, 25.6±0.2, 26.8±0.2, 27.1±0.2 and 27.8±0.2 (obtained at room temperature and CuKαλ=1.5418 Å). In some aspects, Form A has a powder x-ray diffraction pattern comprising 5 or more peaks at 2θ values selected from 6.4±0.2, 6.8±0.2, 9.6±0.2, 13.6±0.2, 14.1±0.2, 14.5±0.2, 14.7±0.2, 15.7±0.2, 18.2±0.2, 18.7±0.2, 19.2±0.2, 19.9±0.2, 20.5±0.2, 21.6±0.2, 22.5±0.2, 23.1±0.2, 24.1±0.2, 24.8±0.2, 25.6±0.2, 26.8±0.2, 27.1±0.2 and 27.8±0.2 (obtained at room temperature and CuKαλ=1.5418 Å).

In some aspects, Form A has a powder x-ray diffraction pattern comprising 2 or more peaks at 2θ values selected from 6.4±0.2, 6.8±0.2, 13.6±0.2, 15.7±0.2, and 21.6±0.2 (obtained at room temperature and CuKαλ=1.5418 Å). In some aspects, Form A has a powder x-ray diffraction pattern comprising 3 or more peaks at 2θ values selected from 6.4±0.2, 6.8±0.2, 13.6±0.2, 15.7±0.2, and 21.6±0.2 (obtained at room temperature and CuKαλ=1.5418 Å). In some aspects, Form A has a powder x-ray diffraction pattern comprising 4 or more peaks at 2θ values selected from 6.4±0.2, 6.8±0.2, 13.6±0.2, 15.7±0.2, and 21.6±0.2 (obtained at room temperature and CuKαλ=1.5418 Å).

In some aspects, Form A has a differential scanning calorimetry thermogram substantially similar to the one as shown in FIG. 2.

In some aspects, Form A has a differential scanning calorimetry thermogram with an endotherm having an onset at about 152° C.

In some aspects, Form A has a thermal gravimetric analysis thermogram substantially similar to the one as shown in FIG. 3.

### III. Compositions

In some aspects, the present disclosure provides compositions comprising Compound A. In some aspects, the present disclosure provides compositions comprising a crystal form of Compound A. In some aspects, the present disclosure provides compositions comprising Form A of Compound A. The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or ignore of the ingredient. Accordingly, the pharmaceutical compositions of the present invention encompass any composition made by mixing a compound of the present invention and a pharmaceutically acceptable carrier. By “pharmaceutically acceptable carrier” it is meant the carrier, diluent or excipient is compatible with the other ingredients of the formulation and not deleterious to the recipient thereof.

In some aspects, the compositions of the disclosure are suitable for oral administration. These compositions can comprise solid, semisolid, gelmatrix or liquid dosage forms suitable for oral administration. As used herein, oral administration includes buccal, lingual, and sublingual administration. Suitable oral dosage forms include, without limitation, tablets, capsules, pills, troches, lozenges, pastilles, cachets, pellets, medicated chewing gum, granules, bulk powders, effervescent or non-effervescent powders or granules, solutions, emulsions, suspensions, solutions, wafers, sprinkles, elixirs, syrups or any combination thereof. In some aspects, compositions of the disclosure suitable for oral administration are in the form of a tablet or a capsule. In some aspects, the compound of the disclosure can be in the form of a capsule. In some aspects, capsules can be immediate release capsules.

The compositions of the disclosure can be in the form of compressed tablets, tablet triturates, chewable lozenges, rapidly dissolving tablets, multiple compressed tablets, or enteric-coating tablets, sugar-coated, or film-coated tablets. Enteric-coated tablets are compressed tablets coated with substances that resist the action of stomach acid but dissolve or disintegrate in the intestine, thus protecting the active ingredients from the acidic environment of the stomach. Enteric-coatings include, but are not limited to, fatty acids, fats, phenylsalicylate, waxes, shellac, ammoniated shellac, and cellulose acetate phthalates. Sugar-coated tablets are compressed tablets surrounded by a sugar coating, which can be beneficial in covering up objectionable tastes or odors and in protecting the tablets from oxidation. Film-coated tablets are compressed tablets that are covered with a thin layer or film of a water-soluble material. Film coatings include, but are not limited to, hydroxyethylcellulose, sodium carboxymethylcellulose, polyethylene glycol 4000, and cellulose acetate phthalate. A film coating can impart the same general characteristics as a sugar coating. Multiple compressed tablets are compressed tablets made by more than one compression cycle, including layered tablets, and press-coated or dry-coated tablets.

In some aspects, the compound of the disclosure can be in the form of a tablet. In some aspects, the compound of the disclosure can be in the form of a compressed tablet. In some aspects, the compound of the disclosure can be in the form of a film-coated compressed tablet. In some aspects, the compositions of the disclosure can be in the form of film-coated compressed tablets.

In some aspects, the compositions of the disclosure can be prepared by fluid bed granulation of the compound of the disclosure with one or more pharmaceutically acceptable carriers, vehicles, and/or excipients. In some aspects, the compositions of the disclosure can be prepared by fluid bed granulation process and can provide a tablet formulation with good flowability, good compressibility, fast dissolution, good stability, and/or minimal to no cracking. In some aspects, the fluid bed granulation process can allow preparation of formulations having high drug loading, such as over 70% or over 75% of a compound of the disclosure.

In some aspects, the compositions of the disclosure can be in the form of soft or hard capsules, which can be made from gelatin, methylcellulose, starch, and/or calcium alginate. The hard gelatin capsule, also known as the dry-filled capsule (DFC), can comprise two sections, one slipping over the other, thus completely enclosing the active ingredient. The soft elastic capsule (SEC) is a soft, globular shell, such as a gelatin shell, which is plasticized by the addition of glycerin, sorbitol, or a similar polyol. In some aspects, soft gelatin shells can contain a preservative to prevent the growth of microorganisms. Suitable preservatives include, but are not limited to, those as described herein, including methyl- and propyl-parabens, sorbic acid, and combinations thereof. The liquid, semisolid, and solid dosage forms provided herein can be encapsulated in a capsule. Suitable liquid and semisolid dosage forms include, but are not limited to, solutions and suspensions in propylene carbonate, vegetable oils, triglycerides, and combinations thereof. The capsules can also be coated as known by those of skill in the art in order to modify or sustain dissolution of the active ingredient.

In some aspects, the compositions of the disclosure can be in liquid or semisolid dosage forms, including emulsions, solutions, suspensions, elixirs, and syrups. In some aspects, the emulsion can be a two-phase system, in which one liquid is dispersed in the form of small globules throughout another liquid, which can be oil-in-water or water-in-oil. Emulsions can include a pharmaceutically acceptable non-aqueous liquids or solvent, emulsifying agent, and preservative. Suspensions can include a pharmaceutically acceptable suspending agent and preservative. Aqueous alcoholic solutions can include a pharmaceutically acceptable acetal, such as a di-(lower alkyl)acetal of a lower alkyl aldehyde (the term “lower” means an alkyl having between 1 and 6 carbon atoms), e.g., acetaldehyde diethyl acetal; and a water-miscible solvent having one or more hydroxyl groups, such as propylene glycol and ethanol. Elixirs can be clear, sweetened, and hydroalcoholic solutions. Syrups can be concentrated aqueous solutions of a sugar, for example, sucrose, and can comprise a preservative. For a liquid dosage form, for example, a solution in a polyethylene glycol can be diluted with a sufficient quantity of a pharmaceutically acceptable liquid carrier, e.g., water, to be measured conveniently for administration.

In some aspects, the compositions of the disclosure for oral administration can be also provided in the forms of liposomes, micelles, microspheres, or nanosystems.

In some aspects, the compositions of the disclosure can be provided as non-effervescent or effervescent, granules and powders, to be reconstituted into a liquid dosage form. Pharmaceutically acceptable carriers and excipients used in the non-effervescent granules or powders can include, but are not limited to, diluents, sweeteners, wetting agents, and mixtures thereof. Pharmaceutically acceptable carriers and excipients used in the effervescent granules or powders can include, but are not limited to, organic acids, a source of carbon dioxide, and mixtures thereof.

Coloring and flavoring agents can be used in all of the above dosage forms. In addition, flavoring and sweetening agents can be especially useful in the formation of chewable tablets and lozenges.

In certain aspects, the compositions of the disclosure can be formulated as immediate or modified release dosage forms, including delayed-, extended, pulsed-, controlled, targeted-, and programmed-release forms.

The compositions of the disclosure can comprise another active ingredient that does not impair the composition's therapeutic or prophylactic efficacy and/or can comprise a substance that augments or supplements the composition's efficacy.

In certain aspects, Compound A or the pharmaceutically acceptable salt and/or solvate thereof, can be administered orally. In some aspects, Compound A, or the pharmaceutically acceptable salt and/or solvate thereof, can be administered in a capsule. In some aspects, Compound A, or the pharmaceutically acceptable salt and/or solvate thereof, can be administered in a tablet.

Compound A, or a pharmaceutically acceptable salt thereof, is typically administered in an admixture with suitable pharmaceutical diluents, excipients, or carriers (collectively referred to herein as pharmaceutical carriers) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with an oral, non-toxic, pharmaceutically acceptable, inert carrier such as lactose, sucrose, dextrose, dextrates, glucose, maltodextrin, mannitol, xylitol, sorbitol, cyclodextrins, calcium phosphate, calcium sulfate, starches, modified starches, methyl cellulose, microcrystalline cellulose, microcellulose, talc and the like; for oral administration in liquid form, the oral drug components can be combined with any oral, non-toxic, pharmaceutically acceptable inert carrier such as ethanol, glycerol, water, and the like. Moreover, when desired or necessary, suitable binders, lubricants, disintegrating agents, glidants, flavoring agents, and coloring agents can also be incorporated into the mixture.

In still other aspects, using standard coating procedures, such as those described in Remingon's Pharmaceutical Sciences, 20th Edition (2000), a film coating is provided around the formulation of Compound A.

Dosage forms (pharmaceutical compositions) suitable for administration can contain from about 1 milligram to about 300 milligrams of active ingredient per dosage unit. In these pharmaceutical compositions the active ingredient will ordinarily be present in an amount of about 0.5−95% by weight based on the total weight of the composition. In some aspects, dosage forms suitable for administration can contain from about 10 to about 240 milligrams of active ingredient per dosage unit. In some aspects, dosage forms suitable for administration can contain from about 30, about 40, about 50, about 60, about 70, about 80, about 90, about 100, about 110, about 120, about 130, about 140, about 150, about 160, about 170, about 180, about 190, about 200, about 210, about 220, about 230, or about 240 mg of active ingredient per dosage unit.

In some aspects, the present disclosure provides pharmaceutical compositions which comprise Compound A, or a pharmaceutically acceptable salt thereof, as described herein, and at least one pharmaceutical acceptable carrier.

In some aspects, the present disclosure provides a pharmaceutical formulation for oral administration comprising:
(a) about 5 wt % to 40 wt % of Compound A;
(b) about 30 wt % to about 90 wt % of a diluent or a mixture of diluents;
(c) about 0 wt % to about 2 wt % of a glidant;
(d) about 2 wt % to about 10 wt % of a disintegrating agent; and
(e) about 0.25 wt % to about 4 wt % of a lubricant.

In some aspects, the Compound A of the pharmaceutical formulation comprises crystalline Form A. In some aspects the pharmaceutical formulation for oral administration is a tablet.

In some aspects, the present disclosure provides a pharmaceutical formulation for oral administration comprising:
(a) about 10 wt % to 30 wt % of Compound A;
(b) about 40 wt % to about 85 wt % of a diluent or a mixture of diluents;
(c) about 0 wt % to about 2 wt % of a glidant;
(d) about 2 wt % to about 10 wt % of a disintegrating agent; and
(e) about 0.25 wt % to about 4 wt % of a lubricant.

In some aspects, the Compound A of the pharmaceutical formulation comprises crystalline Form A. In some aspects the pharmaceutical formulation for oral administration is a tablet.

In some aspects, the diluents described herein are selected from lactose, sucrose, dextrose, dextrates, glucose, maltodextrin, mannitol, xylitol, sorbitol, cyclodextrins, calcium phosphate, calcium sulfate, starches, modified starches, methyl cellulose, microcrystalline cellulose, microcellulose, talc, and combinations thereof. In some aspects, the diluent or diluent mixture is selected from microcrystalline cellulose and anhydrous lactose.

As used herein, the term “glidant” refers a substance that, when added to a powder, improves the flowability of the powder, such as by reducing inter-particle friction. In some aspects, the glidant described herein is selected from silicas, silicon dioxide, CAB-0-SILM-SP, AEROSIL, talc, starch, magnesium aluminum silicates, and combinations thereof. In some aspects, the glidant is silicon dioxide.

In some aspects, the disintegrating agent described herein is selected from natural starch, a pregelatinized starch, a sodium starch, methyl crystalline cellulose, methylcellulose, croscarmellose, croscarmellose sodium, cross-linked sodium carboxymethylcellulose, cross-linked carboxymethylcellulose, cross-linked croscarmellose, cross-linked starch such as sodium starch glycolate, cross-linked polymer such as crospovidone, cross-linked polyvinylpyrrolidone, sodium alginate, a clay, a gum, and combinations thereof. In some aspects, the disintegrating agent is croscarmellose sodium.

In some aspects, the surfactant described herein is selected from sodium lauryl sulfate, sorbitan monooleate, polyoxyethylene sorbitan monooleate, polysorbates, polaxomers, bile salts, glyceryl monostearate, copolymers of ethylene oxide, propylene oxide, and combinations thereof. In some aspects, the surfactant is sodium lauryl sulfate.

In some aspects, the lubricant described herein is selected from stearic acid, calcium hydroxide, talc, corn starch, sodium stearyl fumerate, stearic acid, sodium oleate, sodium stearate, sodium benzoate, sodium acetate, sodium chloride, magnesium stearate, zinc stearate, waxes, and combinations hereof. In some aspects, the lubricant is magnesium stearate.

In some aspects, provided herein is a pharmaceutical formulation for oral administration comprising:
(a) about 5 wt % to 40 wt % of Compound A;
(b) about 15 wt % to about 70 wt % of microcrystalline cellulose and about 15 wt % to about 70 wt % of anhydrous lactose;
(c) about 0 wt % to about 2 wt % of silicon dioxide;
(d) about 2 wt % to about 6 wt % of croscarmellose sodium; and
(e) about 0.25 wt % to about 1.5 wt % of magnesium stearate.

In some aspects, the Compound A of the pharmaceutical formulation comprises crystalline Form A. In some aspects the pharmaceutical formulation for oral administration is a tablet.

In some aspects, provided herein is a pharmaceutical formulation for oral administration comprising:
(a) about 10 wt % to 30 wt % of Compound A;
(b) about 25 wt % to about 70 wt % of microcrystalline cellulose and about 25 wt % to about 70 wt % of anhydrous lactose;
(c) about 0 wt % to about 2 wt % of silicon dioxide;
(d) about 2 wt % to about 6 wt % of croscarmellose sodium; and
(e) about 0.25 wt % to about 1.5 wt % of magnesium stearate.

In some aspects, the Compound A of the pharmaceutical formulation comprises crystalline Form A. In some aspects the pharmaceutical formulation for oral administration is a tablet. In some aspects, tablets can be prepared with the components provided in Tables 1 and/or 2.

TABLE 1
Components of Tablet Formulation

| Ingredient | Range |
|---|---|
| Compound A | 5% to 40% |
| Microcrystalline cellulose | 15% to 70% |
| Anhydrous Lactose | 15% to 70% |
| Silicon dioxide | 0% to 2% |
| Croscarmellose sodium | 2% to 6% |
| Magnesium Stearate | 0.25% to 1.5% |
| Total | Tablet weight range: |
| | 100 mg to 850 mg |

TABLE 2
Components of Tablet Formulation

| Ingredient | Range |
|---|---|
| Compound A | 10% to 30% |
| Microcrystalline cellulose | 25% to 70% |
| Anhydrous Lactose | 25% to 70% |
| Silicon dioxide | 0% to 2% |
| Croscarmellose sodium | 2% to 6% |
| Magnesium Stearate | 0.25% to 1.5% |
| Total | Tablet weight range: |
| | 100 mg to 850 mg |

In some aspects, the pharmaceutical compositions for oral administration can be prespred by direct compression or granulation (dry, wet, or melt granulation).

### IV. Methods of Treatment

The present disclosure provides methods of treating interstitial lung disease by administering Compound A, an LPA₁ antagonist. Lysophospholipids are membrane-derived bioactive lipid mediators. Lysophospholipids include, but are not limited to, lysophosphatidic acid (1-acyl-2-hydroxy-sn-glycero-3-phosphate; LPA), sphingosine 1-phosphate (SIP), lysophosphatidylcholine (LPC), and sphingosylphosphorylcholine (SPC). Lysophospholipids affect fundamental cellular functions that include cellular proliferation, differentiation, survival, migration, adhesion, invasion, and morphogenesis. These functions influence many biological processes that include neurogenesis, angiogenesis, wound healing, immunity, and carcinogenesis.

LPA acts through sets of specific G protein-coupled receptors (GPCRs) in an autocrine and paracrine fashion. LPA binding to its cognate GPCRs (LPA₁, LPA₂, LPA₃, LPA₄, LPA₅, LPA₆) activates intracellular signaling pathways to produce a variety of biological responses.

Lysophospholipids, such as LPA, are quantitatively minor lipid species compared to their major phospholipid counterparts (e.g., phosphatidylcholine, phosphatidylethanolamine, and sphingomyelin). LPA has a role as a biological effector molecule, and has a diverse range of physiological actions such as, but not limited to, effects on blood pressure, platelet activation, and smooth muscle contraction, and a variety of cellular effects, which include cell growth, cell rounding, neurite retraction, and actin stress fiber formation and cell migration. The effects of LPA are predominantly receptor mediated.

Activation of the LPA receptors (LPA₁, LPA₂, LPA₃, LPA₄, LPA₅, LPA₆) with LPA mediates a range of downstream signaling cascades. These include, but are not limited to, mitogen-activated protein kinase (MAPK) activation, adenylyl cyclase (AC) inhibition/activation, phospholipase C (PLC) activation/Ca²⁺ mobilization, arachidonic acid release, Akt/PKB activation, and the activation of small GTPases, Rho, ROCK, Rac, and Ras. Other pathways that are affected by LPA receptor activation include, but are not limited to, cyclic adenosine monophosphate (cAMP), cell division cycle 42/GTP-binding protein (Cdc42), proto-oncogene serine/threonine-protein kinase Raf (c-RAF), proto-oncogene tyrosine-protein kinase Src (c-src), extracellular signal-regulated kinase (ERK), focal adhesion kinase (FAK), guanine nucleotide exchange factor (GEF), glycogen synthase kinase 3b (GSK3b), c-jun amino-terminal kinase (JNK), MEK, myosin light chain II (MLC II), nuclear factor kB (NF-kB), N-methyl-D-aspartate (NMDA) receptor activation, phosphatidylinositol 3-kinase (PI3K), protein kinase A (PKA), protein kinase C (PKC), ras-related C3 botulinum toxin substrate 1 (RAC 1). The actual pathway and realized end point are dependent on a range of variables that include receptor usage, cell type, expression level of a receptor or signaling protein, and LPA concentration. Nearly all mammalian cells, tissues and organs co-express several LPA-receptor subtypes, which indicates that LPA receptors signal in a cooperative manner. LPA₁, LPA₂, and LPA₃ share high amino acid sequence similarity.

LPA is produced from activated platelets, activated adipocytes, neuronal cells, and other cell types. Serum LPA is produced by multiple enzymatic pathways that involve monoacylglycerol kinase, phospholipase A₁, secretory phospholipase A₂, and lysophospholipase D (lysoPLD), including autotaxin. Several enzymes are involved in LPA degradation: lysophospholipase, lipid phosphate phosphatase, and LPA acyl transferase such as endophilin. LPA concentrations in human serum are estimated to be 1−5 μM. Serum LPA is bound to albumin, low-density lipoproteins, or other proteins, which possibly protect LPA from rapid degradation. LPA molecular species with different acyl chain lengths and saturation are naturally occurring, including 1-palmitoyl (16:0), 1-palmitoleoyl (16:1), 1-stearoyl (18:0), 1-oleoyl (18:1), 1-linoleoyl (18:2), and 1-arachidonyl (20:4) LPA. Quantitatively minor alkyl LPA has biological activities similar to acyl LPA, and different LPA species activate LPA receptor subtypes with varied efficacies.

LPA₁ (previously called VZG-1/EDG-2/mrec1.3) couples with three types of G proteins, G_{i/o}, G_{q}, and G_{12/13}. Through activation of these G proteins, LPA induces a range of cellular responses through LPA₁ including but not limited to: cell proliferation, serum-response element (SRE) activation, mitogen-activated protein kinase (MAPK) activation, adenylyl cyclase (AC) inhibition, phospholipase C (PLC) activation, Ca²⁺ mobilization, Akt activation, and Rho activation.

Wide expression of LPA₁ is observed in adult mice, with clear presence in testis, brain, heart, lung, small intestine, stomach, spleen, thymus, and skeletal muscle. Similarly, human tissues also express LPA₁; it is present in brain, heart, lung, placenta, colon, small intestine, prostate, testis, ovary, pancreas, spleen, kidney, skeletal muscle, and thymus.

The term “LPA-dependent”, as used herein, refers to conditions or disorders that would not occur, or would not occur to the same extent, in the absence of LPA.

The term “LPA-mediated,” as used herein, refers to refers to conditions or disorders that might occur in the absence of LPA but can occur in the presence of LPA.

The terms “fibrosis” and “fibrotic disease,” as used herein, refer to conditions that are associated with the abnormal accumulation of cells and/or fibronectin and/or collagen and/or increased fibroblast recruitment and include but are not limited to fibrosis of individual organs or tissues such as the heart, kidney, liver, joints, lung, pleural tissue, peritoneal tissue, skin, cornea, retina, musculoskeletal and digestive tract, such as idiopathic pulmonary fibrosis, scleroderma, and chronic nephropathies.

Exemplary diseases, disorders, or conditions that involve fibrosis include, but are not limited to: lung diseases associated with fibrosis, e.g., idiopathic pulmonary fibrosis, pulmonary fibrosis secondary to systemic inflammatory disease such as rheumatoid arthritis, scleroderma, lupus, cryptogenic fibrosing alveolitis, radiation induced fibrosis, chronic obstructive pulmonary disease (COPD), chronic asthma, silicosis, asbestos induced pulmonary or pleural fibrosis, acute lung injury and acute respiratory distress (including bacterial pneumonia induced, trauma induced, viral pneumonia induced, ventilator induced, non-pulmonary sepsis induced, and aspiration induced); chronic nephropathies associated with injury/fibrosis (kidney fibrosis), e.g., glomerulonephritis secondary to systemic inflammatory diseases such as lupus and scleroderma, diabetes, glomerular nephritis, focal segmental glomerular sclerosis, IgA nephropathy, hypertension, allograft and Alport; gut fibrosis, e.g., scleroderma, and radiation induced gut fibrosis; liver fibrosis, e.g., cirrhosis, alcohol induced liver fibrosis, nonalcoholic steatohepatitis (NASH), biliary duct injury, primary biliary cirrhosis, infection or viral induced liver fibrosis (e.g., chronic HCV infection), and autoimmune hepatitis; head and neck fibrosis, e.g., radiation induced; corneal scarring, e.g., LASIK (laser-assisted in situ keratomileusis), corneal transplant, and trabeculectomy; hypertrophic scarring and keloids, e.g., burn induced or surgical; and other fibrotic diseases, e.g., sarcoidosis, scleroderma, spinal cord injury/fibrosis, myelofibrosis, vascular restenosis, atherosclerosis, arteriosclerosis, Wegener's granulomatosis, mixed connective tissue disease, and Peyronie's disease.

Other diseases, disorders, or conditions where LPA₁ receptors can be involved include atherosclerosis, thrombosis, heart disease, vasculitis, formation of scar tissue, restenosis, phlebitis, COPD (chronic obstructive pulmonary disease), pulmonary hypertension, pulmonary fibrosis, pulmonary inflammation, bowel adhesions, bladder fibrosis and cystitis, fibrosis of the nasal passages, sinusitis, inflammation mediated by neutrophils, and fibrosis mediated by fibroblasts, dermatological disorders including proliferative or inflammatory disorders of the skin such as, atopic dermatitis, bullous disorders, collagenosis, psoriasis, psoriatic lesions, dermatitis, contact dermatitis, eczema, rosacea, wound healing, scarring, hypertrophic scarring, keloids, Kawasaki Disease, rosacea, Sjögren-Larsson Syndrome, and urticaria, respiratory diseases including asthma, adult respiratory distress syndrome and allergic (extrinsic) asthma, non-allergic (intrinsic) asthma, acute severe asthma, chronic asthma, clinical asthma, nocturnal asthma, allergen-induced asthma, aspirin-sensitive asthma, exercise-induced asthma, isocapnic hyperventilation, child-onset asthma, adult-onset asthma, cough-variant asthma, occupational asthma, steroid-resistant asthma, seasonal asthma, seasonal allergic rhinitis, perennial allergic rhinitis, chronic obstructive pulmonary disease, including chronic bronchitis or emphysema, pulmonary hypertension, interstitial lung fibrosis and/or airway inflammation and cystic fibrosis, and hypoxia, and inflammatory/immune disorders including psoriasis, rheumatoid arthritis, vasculitis, inflammatory bowel disease, dermatitis, osteoarthritis, asthma, inflammatory muscle disease, allergic rhinitis, vaginitis, interstitial cystitis, scleroderma, eczema, allogeneic or xenogeneic transplantation (organ, bone marrow, stem cells and other cells and tissues) graft rejection, graft-versus-host disease, lupus erythematosus, inflammatory disease, type I diabetes, pulmonary fibrosis, dermatomyositis, Sjögren's syndrome, thyroiditis (e.g., Hashimoto's and autoimmune thyroiditis), myasthenia gravis, autoimmune hemolytic anemia, multiple sclerosis, cystic fibrosis, chronic relapsing hepatitis, primary biliary cirrhosis, allergic conjunctivitis and atopic dermatitis.

Currently there are two approved treatments for interstitial lung disease: nintedanib and perfenidone, though several compounds are currently in development. In certain aspects of the present disclosure, the subject being administered Compound A, or a pharmaceutically acceptable salt thereof, is receiving concomitant treatment with one or more therapies for interstitial lung disease. In some aspects, the one or more therapies is selected from nintedanib and perfenidone.

In certain aspects, the subject is administered about 100 mg to about 150 mg of Compound A, or an equivalent amount of a pharmaceutically acceptable salt thereof per day. In some aspects, the subject is administered about 110 mg to about 130 mg of Compound A, or an equivalent amount of a pharmaceutically acceptable salt thereof per day. In some aspects the subject is administered about 100, about 105, about 110, about 115, about 120, about 125, about 130, about 135, about 140, about 145, or about 150 mg of Compound A, or an equivalent amount of a pharmaceutically acceptable salt thereof, per day. In some aspects, the subject is administered about 120 mg of Compound A, or an equivalent amount of a pharmaceutically acceptable salt thereof, per day.

In some aspects, the subject is administered Compound A, or a pharmaceutically acceptable salt thereof, once daily. In some aspects, the subject is administered Compound A, or a pharmaceutically acceptable salt thereof, twice daily. In some aspects, the subject is administered Compound A, or a pharmaceutically acceptable salt thereof, three times daily. In some aspects, the subject is administered Compound A, or a pharmaceutically acceptable salt thereof, four times daily. In some aspects, the subject is administered Compound A, or a pharmaceutically acceptable salt thereof, five times daily.

In some aspects, the subject is administered 120 mg of Compound A, or an equivalent amount of a pharmaceutically acceptable salt thereof, once daily. In some aspects, the subject is administered a dose of 60 mg of Compound A, or an equivalent amount of a pharmaceutically acceptable salt thereof, twice daily. In some aspects, the subject is administered a dose of 40 mg of Compound A, or an equivalent amount of a pharmaceutically acceptable salt thereof, three times per day. In some aspects, the subject is administered a dose of 30 mg of Compound A, or an equivalent amount of a pharmaceutically acceptable salt thereof four times per day. In some aspects, the subject is administered a dose of 24 mg, or an equivalent amount of a pharmaceutically acceptable salt thereof, five times per day.

In some aspects, the subject is administered Compound A, or a pharmaceutically acceptable salt thereof, with food. In some aspects, the subject is administered Compound A, or a pharmaceutically acceptable salt thereof, without food.

In some aspects, the subject administered Compound A, or a pharmaceutically acceptable salt thereof, experiences a slower disease progression than an untreated subject. In some aspects, disease progression is measured by the subject's decrease in forced vital capacity (FVC). In some aspects, the subject treated with Compound A, or a pharmaceutically acceptable salt thereof, experiences a smaller decline in forced vital capacity (FVC) after a treatment period compared to an untreated subject. FVC is the amount of air that a subject is able to forcibly exhale from his/her lungs after taking the deepest breath they can. FVC is typically measured using spirometry testing, which involves placing a special mask over the subject's face and having the subject inhale and exhale as forcibly as they can, while the measurements are collected.

In some aspects, disease progression can be measured by the time it takes a subject to experience a disease progression event. In some aspects, the subject administered Compound A, or a pharmaceutically acceptable salt thereof, experiences a greater time to first disease progression event after a treatment period than an untreated subject. In some aspects, the first disease progression event is absolute percentage predicted forced vital capacity (ppFVC) of ≥10% from baseline. An absolute or relative decline in % predicted FVC≥10% is associated with mortality. In some aspects, the subject treated with Compound A, or a pharmaceutically acceptable salt thereof, experiences a greater time to absolute percentage predicted forced vital capacity (ppFVC) of ≥10% from baseline than an untreated subject.

In some aspects, the first disease progression event is acute exacerbation (e.g., sudden worsening) of his/her lung fibrosis. In some aspects, the subject treated with Compound A, or a pharmaceutically acceptable salt thereof, experiences a greater time to acute exacerbation of lung fibrosis than an untreated subject.

In some aspects, the first disease progression event is respiratory hospitalization. In some aspects, the subject treated with Compound A, or a pharmaceutically acceptable salt thereof, experiences a greater time to respiratory hospitalization than an untreated subject.

In some aspects, the first disease progression event is lung fibrosis-related hospitalization. In some aspects, the subject treated with Compound A, or a pharmaceutically acceptable salt thereof, experiences a greater time to lung fibrosis-related hospitalization than an untreated subject.

In some aspects, the first disease progression event is lung transplantation. In some aspects, the subject treated with Compound A, or a pharmaceutically acceptable salt thereof, experiences a greater time to lung transplantation than an untreated subject.

In some aspects, the first disease progression event is mortality. In some aspects, the subject treated with Compound A, or a pharmaceutically acceptable salt thereof, experiences a greater time to all-cause mortality than an untreated subject.

In some aspects, the subject experiences a greater time to first disease progression event after a treatment period than an untreated subject, wherein the first disease progression event is selected from:
absolute percentage predicted forced vital capacity (ppFVC) of ≥10% from baseline;
acute exacerbation of lung fibrosis;
respiratory hospitalization;
lung transplantation; and
all-cause mortality.

In some aspects, the subject experiences a greater time to first disease progression event after a treatment period than an untreated subject, wherein the first disease progression event is selected from:
absolute predicted forced vital capacity (ppFVC) decline of ≥10% from baseline;
acute exacerbation of lung fibrosis;
lung fibrosis-related hospitalization; and
all-cause mortality.

In some aspects, disease progression is measured by a change in score in the Living with Pulmonary Fibrosis (L-PF) questionnaire. The Living with Pulmonary Fibrosis (L-PF) questionnaire assesses symptoms and quality of life in patients with fibrosing interstitial lung diseases (ILDs). Its Dyspnoea and Cough domains, whose items' responses are based on a 24-hour recall, have scores ranging from 0 to 100, with higher scores indicating greater symptom severity (see, for example, Swigris J J, el al. BMJ Open Resp Res 2022; 9:e001167. doi: 10.1136/bmjresp-2021−001167). In some aspects, the subject administered Compound A, or a pharmaceutically acceptable salt thereof, experiences a smaller increase in cough domain score as measured by the Living with Pulmonary Fibrosis (L-PF) questionnaire over a treatment period than an untreated subject. In some aspects, the subject experiences a smaller increase in dyspnea score as measured by the Living with Pulmonary Fibrosis (L-PF) questionnaire over a treatment period than an untreated subject.

### EXAMPLES

### Example 1: Preparation of Crystalline Form A of Compound A

Crystal forms can be prepared by a variety of methods, including for example, crystallization or recrystallization from a suitable solvent, sublimation, growth from a melt, solid state transformation from another phase, crystallization from a supercritical fluid, and jet spraying. Techniques for crystallization or recrystallization of co-crystal forms from a solvent mixture include, for example, evaporation of the solvent, decreasing the temperature of the solvent mixture, crystal seeding a supersaturated solvent mixture of the molecule and/or salt, freeze drying the solvent mixture, and addition of antisolvents (countersolvents) to the solvent mixture.

For crystallization techniques that employ solvent, the choice of solvent or solvents is typically dependent upon one or more factors, such as solubility of the compound, crystallization technique, and vapor pressure of the solvent. Combinations of solvents can be employed, for example, the compound can be solubilized into a first solvent to afford a solution, followed by the addition of an antisolvent to decrease the solubility of the compound in the solution and to afford the formation of crystals. An antisolvent is a solvent in which the compound has low solubility.

In one method to prepare crystals, a compound is suspended and/or stirred in a suitable solvent to afford a slurry, which can be heated to promote dissolution. The term “slurry”, as used herein, means a saturated solution of the compound, which can also contain an additional amount of the compound to afford a heterogeneous mixture of the compound and a solvent at a given temperature.

Seed crystals can be added to any crystallization mixture to promote crystallization. Seeding can be employed to control growth of a particular polymorph or to control the particle size distribution of the crystalline product. Accordingly, calculation of the amount of seeds needed depends on the size of the seed available and the desired size of an average product particle as described, for example, in “Programmed Cooling of Batch Crystallizers,” J. W. Mullin and J. Nyvlt, Chemical Engineering Science, 1971, 26, 369−377. In general, seeds of small size are needed to control effectively the growth of crystals in the batch. Seed of small size can be generated by sieving, milling, or micronizing of large crystals, or by micro-crystallization of solutions. Care should be taken that milling or micronizing of crystals does not result in any change in crystallinity form the desired crystal form (i.e., change to amorphous or to another polymorph).

A cooled crystallization mixture can be filtered under vacuum, and the isolated solids can be washed with a suitable solvent, such as cold recrystallization solvent, and dried under a nitrogen purge to afford the desired crystalline form. The isolated solids can be analyzed by a suitable spectroscopic or analytical technique, such as solid state nuclear magnetic resonance, differential scanning calorimetry, x-ray powder diffraction, or the like, to assure formation of the preferred crystalline form of the product. The resulting crystalline form is typically produced in an amount of greater than about 70 weight % isolated yield, preferably greater than 90 weight % isolated yield, based on the weight of the compound originally employed in the crystallization procedure. The product can be comilled or passed through a mesh screen to delump the product, if necessary.

The presence of more than one polymorph in a sample can be determined by techniques such as powder x-ray diffraction (PXRD) or by Raman or IR spectroscopy solid state nuclear magnetic resonance spectroscopy. For example, the presence of extra peaks in the comparison of an experimentally measured PXRD pattern with a simulated PXRD pattern can indicate more than one polymorph in the sample. The simulated PXRD can be calculated from single crystal x-ray data. see Smith, D. K., “A FORTRAN Program for Calculating X-Ray Powder Diffraction Patterns,” Lawrence Radiation Laboratory, Livermore, California, UCRL-7196 (April 1963).

The crystalline form of Compound A according to the invention can be characterized using various techniques, the operation of which are well known to those of ordinary skill in the art. The forms can be characterized and distinguished using single crystal x-ray diffraction, which is based on unit cell measurements of a single crystal of form at a fixed analytical temperature. A detailed description of unit cells is provided in Stout & Jensen, X-Ray Structure Determination: A Practical Guide, Macmillan Co., New York (1968), Chapter 3, which is herein incorporated by reference. Alternatively, the unique arrangement of atoms in spatial relation within the crystalline lattice can be characterized according to the observed fractional atomic coordinates. Another means of characterizing the crystalline structure is by powder x-ray diffraction analysis in which the diffraction profile is compared to a simulated profile representing pure powder material, both run at the same analytical temperature, and measurements for the subject form characterized as a series of 2q values (usually four or more).

Other means of characterizing the form can be used, such as solid state nuclear magnetic resonance (SSNMR), differential scanning calorimetry, thermogravimetric analysis and FT-Raman and FT-IR. These techniques can also be used in combination to characterize the subject form. In addition to the techniques specifically described herein, the presence of a particular crystalline form can be determined by other suitable analytical methods.

### Example 1A

150 mg Compound A was dissolved in 1.5 mL tetrahydrofuran (THF) at 20° C. 0.5 mL of this solution was subjected to flash evaporation using a centrifugal evaporator to yield solids of Form A. 0.5 mL of the same solution was subjected to slow evaporation at 20° C. to yield solids of Form A.

### Example 1B

200 mg Compound A was dissolved in 1 mL dichloromethane (DCM) at 20° C. 0.5 mL of this solution was subjected to flash evaporation using a centrifugal evaporator to yield solids of Form A. 0.5 mL of the same solution was subjected to slow evaporation at 20° C. to yield solids of Form A.

### Example 1C

100 mg of Compound A was dissolved in 0.5 mL of THE at 50° C. and kept stirring at 20° C. 0.5 mL of water was added to the clear solution, which resulted in solids of Form A.

### Example 1D

100 mg of Compound A was dissolved in 1 mL of 2-methyl THE at 50° C. and kept stirring at 20° C. 1 mL of n-heptane was added to the clear solution, which resulted in solids of Form A.

### Example 1E

A solution of Compound A in tert-Amyl alcohol (t-AmOH) was concentrated to 4 L/kg under vacuum, then was charged with 15 L/kg DCM and 10 L/kg water. Layers were split and the DCM layer was concentrated to 4 L/kg under vacuum. The DCM layer was charged with 8−10 L/kg ethyl acetate (EtOAc) and then concentrated to 4 L/kg under vacuum. An additional 8−10 L/kg EtOAc was charged then concentrated to 4 L/kg under vacuum. 6−8 L/kg EtOAc was charged and warmed to 70−83° C. until complete dissolution. The resulting slurry was cooled to 0−10° C. over at least 2 hours, followed by aging for at least an additional 12 hours. The slurry was filtered. The wet cake was washed with 3−5 L/kg EtOAc and dried under vacuum at 55−60° C. to yield solids of Form A.

### Example 1F

A solution of Compound A in t-AmOH was concentrated to 4 L/kg at 55° C. under vacuum, was then charged with 5 L/kg 2-propanol (IPA) and concentrated to 4 L/kg at 55° C. under vacuum. This process was repeated two additional times with 2×5 L/kg IPA. The batch was cooled to 30° C., was then charged with 1.3 L/kg water and heated to 45−55° C. The resulting warm solution was polish filtered and cooled to 30° C. 1 wt % Form A seeds were charged, followed by 2 L/kg water. After at least 6 hours, an additional 8.7 L/kg water was charged. The resulting slurry was cooled to 20° C. over at least 30 minutes, and the slurry was aged at least 3 hours. The solids were filtered and the wet cake was sequentially washed with 3 L/kg water:IPA:t-AmOH mixture (11:3:1 by volume) and 3 L/kg water, and dried under vacuum at 50−60° C. to solids of Form A.

### Example 1G

To a solution of isopropyl (1S,3S)-3-((2-methyl-6-(1-methyl-5-(((methyl(propyl)carbamoyl)oxy) methyl)-1H-1,2,3-triazol-4-yl)pyridin-3-yl)oxy)cyclohexane-1-carboxylate (500 mg, 1.025 mmol) in 1:1 THF/MeOH (10 mL) was added aqueous LiOH (1.538 mL of a 2M solution, 3.08 mmol). The reaction mixture was stirred at 50° C. for 1 h, then was cooled to RT, and organic volatiles were removed in vacuo. The concentrated solution was washed with EtOAc, then was acidified to pH ˜6−7 (1N aq HCl). This aqueous layer was extracted with EtOAc several times. The combined organic extracts were washed with water, dried (MgSO₄) and the MgSO₄ was filtered off. The EtOAc solution was concentrated in vacuo to yield solids of Form A.

The analytical data for the crystalline Compound A described herein were obtained using the following procedures.

### Single Crystal

Single crystal X-ray data were collected using a Bruker X8 Kappa diffractometer equipped with an APEX II CCD detector and a MICROSTAR microfocus rotating anode X-ray generator of monochromatic Cu Kα radiation. The single crystal was at room temperature during data collection.

Indexing and processing of the measured intensity data were carried out with the APEX2 program suite (Bruker AXS, Inc., 5465 East Cheryl Parkway, Madison, WI 53711 USA). The final unit cell parameters were determined using the full data set. The structures were solved by direct methods and refined by full-matrix least-squares approach using the SHELXTL software package (G. M. Sheldrick, SHELXTL v6.14, Bruker AXS, Madison, WI USA). Structure refinements involved minimization of the function defined by Σw(|Fₒ|−|F_{c}|)², where w is an appropriate weighting factor based on errors in the observed intensities, Fₒ is the structure factor based on measured reflections, and F_{c} is the structure factor based on calculated reflections. Agreement between the refined crystal structure model and the experimental X-ray diffraction data is assessed by using the residual factors R=Σ∥Fₒ−|F_{c}∥/Σ|Fₒ| and

$$w R = {[ Σ {w ( \vert {F}_{o} \vert - \vert {F}_{c} \vert )}^{2} / Σ w \vert {F}_{o} \vert ]}^{1 / 2} .$$

Difference Fourier maps were examined at all stages of refinement. All non-hydrogen atoms were refined with anisotropic thermal displacement parameters. Hydrogen atoms were introduced using idealized geometry with isotropic temperature factors and included in structure factor calculations with fixed parameters.

### Powder X-Ray Diffraction (PXRD)

PXRD diffractogram was acquired on a Bruker D8 Advance system using Cu Kα (40 kV/40 mA) radiation and a step size of 0.03° 2q and LynxEye detector, over a 2θ range of 2−40°. Configuration on the incident beam side: Göebel mirror, mirror exit slit (0.2 mm), 2.5 deg Soller slits, beam knife. Configuration on the diffracted beam side: anti-scatter slit (8 mm) and 2.5 deg. Soller slits. Sample was mounted flat on zero-background Si wafers.

### Differential Scanning Calorimetry (DSC)

DSC was conducted with a TA Instruments Q2000 differential scanning calorimeter equipped with an autosampler and a refrigerated cooling system under 40 mL/min N₂ purge for the Q2000. DSC thermogram was obtained at 15° C./min in crimped A1 pan.

### Thermal Gravimetric Analysis (TGA)

TGA thermograms were obtained with a TA Instruments Q500 thermogravimetric analyzer under 40 mL/min N₂ purge for balance and 60 mL/min for sample in A1 pan. TGA thermogram was obtained at 15° C./min.

### Moisture Sorption Isotherms

Moisture sorption isotherms were collected in a TA Instrument VTI-SA+Vapor Sorption Analyzer using approximately 270 mg of sample in a 250 μL ceramic pan. The sample was dried at 30° C. until the loss rate of 0.005 wt % (min was obtained for 10 minutes. The sample was tested at 25° C. and 4, 5, 15, 25, 35, 45, 50, 65, 75, 85, and 95% RH. Equilibration at each RH was reached when the rate of 0.01 wt %/min for 35 minutes was achieved or a maximum of 600 minutes.

### Example 2. A Double-Blind, Placebo-Controlled, Randomized, Single and Multiple Ascending Dose Study of the Safety, Pharmacokinetics, and Exploratory Pharmacodynamics of Oral Administration of Compound A in Healthy Participants

The study was performed in 3 parts in 112 healthy male and female participants, including 24 Japanese participants. Female participants were to be not of childbearing potential. The present study was designed to evaluate the safety and tolerability, PK, and exploratory PD of an oral suspension of Compound A. Parts A and B (Cohort B1 only) enrolled healthy participants and was conducted at a site in The Netherlands and Part B (Cohort B2 to B5) enrolled healthy non-Japanese participants and was conducted at a site in the United Kingdom (UK). Part C enrolled healthy Japanese participants and was conducted at the same site in the UK.

Part A was an SAD study in a planned number of 6 sequential dose escalation cohorts (3, 10, 30, 100, 150, or 250 mg) of 8 healthy participants (6 active+2 placebo) each, under fasted conditions. Eligible participants in Part A (excluding those in Cohort A7 [food effect (FE)/pH cohort]) received a single administration of study drug (Compound A or placebo) under fasted conditions. In Cohort A1 to Cohort A6, sentinel dosing was employed (1 active+1 placebo, followed after 48 hours by the remaining participants of the cohort [5 active+1 placebo]). However, in Cohort A6 (250 mg) only the 2 sentinel participants were dosed before discontinuing further enrollment, due to dose-limiting events observed in a sentinel participant. In addition to the 6 ascending dose cohorts, a cohort of 6 healthy participants (Cohort A7; 6 active, 100 mg Compound A) was included to assess the effect of food and pH on the bioavailability of Compound A in a 3-period, open-label, fixed-sequence, crossover design (FE/pH cohort).

Part B was a MAD study in a planned number of 6 sequential dose escalation cohorts (10 mg QD, 30 mg QD, 30 mg BID, 60 mg BID, 125 mg BID, and ≤250 mg BID) of 8 healthy participants (6 active+2 placebo) each, under fasted conditions. However, Cohort B6 (≤250 mg BID) was cancelled because sufficient safety and PK data had been obtained at doses up to 125 mg BID. Eligible participants received study drug (Compound A or placebo) administered orally for 14 days.

Part C was a MAD study in 3 sequential dose escalation cohorts (30 mg BID, 60 mg BID, and 90 mg BID) of 8 healthy Japanese participants (6 active+2 placebo) each. Eligible participants in Cohorts C1 to C3 received study drug (Compound A or placebo) administered orally for 14 days.

Physical examinations, vital sign measurements (including orthostasis testing in Cohort A6, Cohort B3 to B5, and C1 to C3 at selected time points), 12-lead electrocardiogram (ECG), and clinical laboratory evaluations were performed at selected times throughout the study. In addition, Holter monitoring was performed for the first 24 hours post dose on Day 1 in Part A (not in FE/pH cohort), and also for the first 24 hours post dose on Day 1 and Day 14 in Parts B and C. Participants were closely monitored for adverse events (AEs). Blood samples were collected for up to 14 days (follow-up visit) after (last) study drug administration for PK analysis. In addition, blood and urine were collected for exploratory biomarker assessments and for biobanking of samples for potential analyses. Urine was collected for potential PK analysis for up to 96 hours after a single dose of Compound A or placebo was administered in the SAD part, and for up to 24 hours after the first (morning) dose of study drug on Day 1 and for up to 24 hours after the last (morning) dose of study drug on Day 14 in the MAD parts.

Compound A was generally safe and well tolerated following single and multiple dose oral administration to healthy participants. It was observed that Compound A was associated with reversible reductions in BP that generally reached a maximum 4 to 8 hours after dosing and were not associated with meaningful changes in heart rate. BP reductions were mostly asymptomatic.

In the SAD cohorts, Compound A decreased SBP and DBP dose dependently, reversibly, and mostly asymptomatically with minimal effect on heart rate. Changes in blood pressure are shown in Tables 3 and 4 below.

TABLE 3
Maximum of Mean (±SD) Reduction in Systolic and Diastolic Blood Pressure
from Baseline and Corresponding Time in SAD within 12 Hours Post Dose

| | SBP (mmHg) | | | DBP (mmHg) | | |
|---|---|---|---|---|---|---|
| Dose (mg) | Baseline | Reduction | Time | Baseline | Reduction | Time |
| (Cohort) | (mmHg) | (mmHg) | (h) | (mmHg) | (mmHg) | (h) |
| Placebo | 124.3 ± 15.43 | 0.5 ± 10.16 | 12 | 70.5 ± 6.38 | 4.5 ± 7.69 | 5 |
| 3 (A1) | 124.7 ± 13.00 | 5.2 ± 3.43 | 2 | 71.2 ± 8.28 | 6.7 ± 3.14 | 6 |
| | | | | | 6.7 ± 5.82^{a} | 12 |
| 10 (A2) | 119.0 ± 10.43 | 9.5 ± 16.13 | 8 | 71.0 ± 7.82 | 8.8 ± 9.91 | 6 |
| 30 (A3) | 117.7 ± 11.38 | 11.7 ± 7.61 | 4 | 70.3 ± 4.46 | 6.8 ± 1.72 | 5 |
| 100 (A4) | 116.5 ± 7.09 | 15.5 ± 11.59 | 2 | 65.7 ± 7.81 | 9.7 ± 8.64 | 5 |
| 100 fasted | 123.8 ± 8.98 | 25.8 ± 9.54 | 8 | 74.8 ± 4.96 | 12.5 ± 7.06 | 5 |
| (A7) | | | | | | |
| 100 fasted + | 121.7 ± 9.75 | 19.3 ± 17.41 | 6 | 73.3 ± 7.39 | 11.8 ± 8.40 | 6 |
| famotidine | | | | | | |
| (A7) | | | | | | |
| 100 fed (A7) | 115.5 ± 7.37 | 18.5 ± 12.94 | 6 | 68.2 ± 3.54 | 12.7 ± 4.18 | 5 |
| 150 (A5) | 116.3 ± 11.34 | 16.5 ± 8.41 | 6 | 63.7 ± 11.62 | 12.7 ± 5.61 | 6 |

DBP = diastolic blood pressure;
SAD = single ascending dose;
SBP = systolic blood pressure;
SD = standard deviation
^{a}Identical maximum reduction to an earlier measurement
Note:
Baseline is defined as the last observation recorded before the first study drug administration in each period.
Time (h) is the time since the last morning study drug administration.

TABLE 4
Mean (±SD) Systolic and Diastolic Blood Pressure in SAD within 24 Hours Post Dose

| | | | | | | | | 100 mg | |
|---|---|---|---|---|---|---|---|---|---|
| | | | | | | | 100 mg | Fasted + | 100 mg |
| | Placebo | 3 mg | 10 mg | 30 mg | 100 mg | 150 mg | Fasted | Famotidine | Fed |
| N | 11 | 6 | 6 | 6 | 6 | 6 | 6 | 6 | 6 |

SBP

| | | | | | | | | | |
|---|---|---|---|---|---|---|---|---|---|
| Baseline | 124.3 | 124.7 | 119.0 | 117.7 | 116.5 | 116.3 | 123.8 | 121.7 | 115.5 |
| | (15.43) | (13.00) | (10.43) | (11.38) | (7.09) | (11.34) | (8.98) | (9.75) | (7.37) |
| D 1 2 h | 125.1 | 119.5 | 112.2 | 107.5 | 101.0 | 105.0 | 107.5 | 111.3 | 100.5 |
| | (10.19) | (13.00) | (7.19) | (11.57) | (11.14) | (14.04) | (12.06) | (13.63) | (16.81) |
| D 1 4 h | 124.9 | 120.3 | 110.2 | 106.0 | 101.3 | 101.5 | 103.8 | 106.5 | 104.2 |
| | (10.87) | (10.33) | (6.24) | (10.16) | (13.29) | (7.74) | (10.30) | (14.46) | (11.27) |
| D 1 8 h | 124.8 | 124.5 | 109.5 | 113.8 | 105.0 | 107.2 | 98.0 | 105.0 | 100.3 |
| | (11.11) | (10.37) | (9.22) | (17.70) | (7.13) | (12.86) | (10.77) | (13.19) | (17.13) |
| D 2 24 h | 121.3 | 118.8 | 118.5 | 120.2 | 114.2 | 114.7 | 115.2 | 118.7 | 112.8 |
| | (18.24) | (6.59) | (12.96) | (12.69) | (6.11) | (12.18) | (6.85) | (9.48) | (5.67) |

DBP

| | | | | | | | | | |
|---|---|---|---|---|---|---|---|---|---|
| Baseline | 70.5 | 71.2 | 71.0 | 70.3 | 65.7 | 63.7 | 74.8 | 73.3 | 68.2 |
| | (6.38) | (8.28) | (7.82) | (4.46) | (7.81) | (11.62) | (4.96) | (7.39) | (3.54) |
| D 1 2 h | 72.2 | 68.3 | 67.7 | 66.2 | 59.0 | 58.2 | 67.2 | 67.2 | 57.2 |
| | (7.88) | (9.83) | (3.27) | (3.19) | (5.97) | (7.36) | (5.00) | (4.75) | (5.49) |
| D 1 4 h | 70.3 | 69.5 | 68.7 | 65.2 | 58.7 | 56.0 | 64.2 | 65.3 | 58.2 |
| | (7.39) | (10.67) | (4.41) | (4.71) | (7.61) | (4.69) | (2.86) | (4.93) | (7.68) |
| D 1 8 h | 69.6 | 68.0 | 63.0 | 65.2 | 56.3 | 57.8 | 63.0 | 63.2 | 58.8 |
| | (8.44) | (4.90) | (4.73) | (4.83) | (7.58) | (5.60) | (6.66) | (4.49) | (5.31) |
| D 2 24 h | 70.1 | 68.7 | 71.7 | 71.0 | 66.0 | 66.2 | 69.3 | 71.7 | 69.3 |
| | (5.86) | (5.32) | (7.42) | (4.73) | (10.71) | (10.72) | (2.34) | (4.63) | (3.72) |

DBP = diastolic blood pressure,
SAD = single ascending dose;
SBP = systolic blood pressure;
SD = standard deviation
Note:
Baseline is defined as the last observation recorded before the first study drug administration in each period.
Time (h) is the time since the last morning study drug administration.

In the MAD cohorts, reversible reductions in mean SBP and DBP occurred in all groups except for the placebo QD group, which showed no change in BP. In the QD groups, reductions generally occurred in a Compound A dose-dependent manner. In the BID groups, both placebo and Compound A treatment resulted in reductions in BP, and there were no clear differences between placebo and the Compound A groups. There were no clear changes in the magnitude of BP reductions with repeated dosing over the 14-day treatment period. In the Japanese MAD cohorts, reversible reductions in mean BP occurred in all groups, including placebo. There was no clear Compound A dose-dependent BP reduction trend. Changes in blood pressure can be found in Tables 5−7 shown below.

TABLE 5
Maximum of Mean (±SD) Reduction in Systolic and Diastolic Blood
Pressure from Baseline and Corresponding Time - Part B (MAD Cohorts)

| | SBP | | | DBP (mmHg) | | |
|---|---|---|---|---|---|---|
| | Baseline | Reduction | Time | Baseline | Reduction | Time |
| Dose (mg) | (mmHg) | (mmHg) | (Day-h) | (mmHg) | (mmHg) | (Day-h) |
| Placebo QD | 113.8 ± 8.14 | 6.0 ± 6.78 | 9-0 | 66.3 ± 5.50 | 0.5 ± 5.69 | 12-0 |
| | | | | | 0.5 ± 7.23 | 14-2 |
| 10 QD (B1) | 115.2 ± 14.03 | 6.7 ± 11.83 | 2-0 | 69.0 ± 11.37 | 6.8 ± 2.64 | 1-5 |
| | | 6.7 ± 11.36 | 13-0 | | | |
| 30 QD (B2) | 112.2 ± 13.96 | 10.7 ± 7.06 | 14-8 | 70.7 ± 7.76 | 8.8 ± 4.62 | 14-5 |
| Placebo BID | 124.8 ± 8.75 | 20.2 ± 10.11 | 13-0 | 64.5 ± 6.16 | 8.7 ± 10.63 | 14-5 |
| 30 BID (B3) | 121.3 ± 11.43 | 15.8 ± 10.19 | 1-5 | 70.0 ± 3.46 | 11.2 ± 4.58 | 1-5 |
| 60 BID (B4) | 123.2 ± 8.68 | 17.5 ± 11.31 | 17-0 | 68.2 ± 3.82 | 8.3 ± 6.89 | 3-4 |
| 125 BID (B5) | 118.5 ± 7.84 | 14.5 ± 5.86 | 9-0 | 69.7 ± 3.83 | 14.7 ± 5.68 | 12-12 |

BID = twice daily;
DBP = diastolic blood pressure;
MAD = multiple ascending dose;
QD = once daily;
SBP = systolic blood pressure;
SD = standard deviation
Note:
Baseline is defined as the last observation recorded before the first study drug administration in each period.
Time (h) is the time since the last morning study drug administration

TABLE 6
Mean (±SD) Systolic and Diastolic Blood Pressure in MAD within 24 Hours Post First and Last Dose

| | Placebo QD | 10 mg QD | 30 mg QD | Placebo BID | 30 mg BID | 60 mg BID | 125 mg BID |
|---|---|---|---|---|---|---|---|
| N | 4 | 6 | 6 | 6 | 6 | 6 | 6 |

SBP

| | | | | | | | |
|---|---|---|---|---|---|---|---|
| Baseline | 113.8 | 115.2 | 112.2 | 124.8 | 121.3 | 123.2 | 118.5 |
| | (8.14) | (14.03) | (13.96) | (8.75) | (11.43) | (8.68) | (7.84) |
| D 1 2 h | 118.8 | 112.2 | 104.8 | 120.3 | 106.5 | 110.2 | 112.2 |
| | (10.40) | (18.74) | (10.23) | (5.79) | (6.72) | (5.81) | (12.45) |
| D 1 4 h | 115.5 | 109.5 | 102.3 | 118.7 | 107.2 | 106.7 | 112.2 |
| | (12.56) | (18.06) | (6.59) | (7.92) | (6.59) | (7.55) | (11.57) |
| D 1 8 h | 117.3 | 112.8 | 107.2 | 111.3 | 106.3 | 109.3 | 107.3 |
| | (11.00) | (20.93) | (9.56) | (8.82) | (5.92) | (10.15) | (14.43) |
| D 2 24 h | 114.3 | 108.5 | 114.3 | 114.2 | 114.7 | 118.8 | 118.8 |
| | (5.91) | (17.35) | (8.41) | (10.68) | (6.15) | (9.35) | (18.74) |
| D 14 312 h | 113.0 | 112.8 | 110.0 | 108.5 | 119.7 | 111.8 | 111.3 |
| | (11.92) | (15.89) | (14.82) | (4.89) | (8.21) | (7.52) | (8.48) |
| D 14 314 h | 111.3 | 113.5 | 101.7 | 110.2 | 109.7 | 110.5 | 116.0 |
| | (8.10) | (16.13) | (5.75) | (4.62) | (12.26) | (5.92) | (8.51) |
| D 14 317 h | 119.0 | 114.5 | 102.3 | 113.2 | 114.3 | 114.8 | 116.3 |
| | (15.64) | (18.80) | (10.48) | (7.19) | (5.96) | (11.57) | (9.35) |
| D 14 320 h | 112.5 | 111.2 | 101.5 | 109.5 | 110.0 | 115.5 | 114.5 |
| | (12.37) | (17.61) | (9.95) | (8.24) | (7.21) | (7.56) | (13.62) |
| D 15 336 h | 113.3 | 110.8 | 107.3 | 110.5 | 116.3 | 114.5 | 111.3 |
| | (13.23) | (18.05) | (9.67) | (4.76) | (5.32) | (6.72) | (10.75) |

DBP

| | | | | | | | |
|---|---|---|---|---|---|---|---|
| Baseline | 66.3 | 69.0 | 70.7 | 64.5 | 70.0 | 68.2 | 69.7 |
| | (5.50) | (11.37) | (7.76) | (6.16) | (3.46) | (3.82) | (3.83) |
| D 1 2 h | 70.5 | 70.2 | 66.5 | 67.7 | 63.3 | 64.5 | 62.3 |
| | (5.20) | (15.96) | (4.97) | (5.92) | (4.37) | (3.33) | (7.12) |
| D 1 4 h | 69.5 | 65.8 | 67.8 | 67.2 | 62.7 | 66.0 | 64.7 |
| | (4.43) | (13.32) | (6.59) | (4.02) | (3.98) | (6.23) | (1.97) |
| D 1 8 h | 70.0 | 68.7 | 66.0 | 60.5 | 63.2 | 64.7 | 64.7 |
| | (7.02) | (12.31) | (7.92) | (10.15) | (1.94) | (2.94) | (4.46) |
| D 2 24 h | 67.8 | 65.3 | 67.3 | 62.7 | 66.7 | 65.7 | 66.3 |
| | (4.19) | (11.06) | (9.14) | (7.76) | (5.82) | (4.97) | (2.94) |
| D 14 312 h | 70.0 | 69.3 | 63.3 | 58.7 | 68.7 | 64.8 | 62.0 |
| | (5.48) | (10.23) | (4.76) | (8.91) | (4.80) | (6.71) | (7.90) |
| D 14 314 h | 65.8 | 67.5 | 63.5 | 62.0 | 63.8 | 64.0 | 65.0 |
| | (6.85) | (12.69) | (3.08) | (6.99) | (3.43) | (6.84) | (3.29) |
| D 14 317 h | 66.5 | 64.3 | 61.8 | 55.8 | 61.0 | 62.5 | 59.2 |
| | (7.68) | (8.09) | (5.38) | (7.65) | (5.37) | (5.17) | (7.63) |
| D 14 320 h | 70.0 | 67.5 | 63.2 | 57.7 | 617 | 63.0 | 61.8 |
| | (8.04) | (10.45) | (5.12) | (8.78) | (3.78) | (4.15) | (6.11) |
| D 15 336 h | 66.5 | 66.8 | 62.3 | 60.7 | 65.3 | 64.5 | 63.0 |
| | (8.89) | (9.83) | (5.28) | (5.99) | (3.88) | (3.67) | (6.00) |

BID = twice daily;
DBP = diastolic blood pressure;
MAD = multiple dose;
QD = once daily;
SBP = systolic blood pressure;
SD = standard deviation
Note:
Baseline is defined as the last observation recorded before the first study drug administration in each period.
Time (h) is the time since the last morning study drug administration.

TABLE 7
Maximum of Mean (±SD) Reduction in Systolic and Diastolic Blood Pressure
from Baseline and Corresponding Time - Part C (MAD Japanese Cohorts)

| | SBP | | | DBP (mmHg) | | |
|---|---|---|---|---|---|---|
| | Baseline | Reduction | Time | Reduction | Baseline | Time |
| Dose (mg) | (mmHg) | (mmHg) | (Day-h)) | (mmHg) | (mmHg) | (Day-h) |
| Placebo BID | 118.7 ± 8.57 | 12.5 ± 9.20 | 2-16 | 76.5 ± 7.94 | 11.2 ± 7.36 | 2-16 |
| 30 BID (C1) | 117.5 ± 5.82 | 15.0 ± 6.84 | 1-4 | 72.2 ± 3.87 | 8.8 ± 5.12 | 14-6 |
| 60 BID (C2 | 114.8 ± 10.26 | 13.0 ± 8.22 | 2-16 | 73.8 ± 6.18 | 8.2 ± 10.40 | 4-16 |
| 90 BID (C2) | 111.5 ± 8.12 | 11.8 ± 8.47 | 1-6 | 70.7 ± 7.58 | 8.0 ± 8.22 | 1-6 |

BID = twice daily; DBP = diastolic blood pressure; MAD = multiple ascending dose; SBP = systolic blood pressure.
Note:
Baseline is defined as the last observation recorded before the first study drug administration in each period.
Time (h) is the time since the last morning study drug administration.

### Example 3. A Double-Blind, Placebo-Controlled, Randomized, Single and Multiple Ascending Dose Study of the Safety, Tolerability and Pharmacokinetics of Oral Administration of Compound a in Healthy Chinese Participants

Single dose of 60 mg and multiple doses of 60 mg BID are selected for this China PK bridging study to allow PK, safety and tolerability for clinically relevant doses. It will investigate the effect of genetic variations in genes associated with drug absorption, distribution, metabolism, excretion, and transport on the PK of Compound A in healthy Chinese participants.

### Single Dose

On Day 1, eligible subjects will be randomized in a 3:1 ratio to a single oral dose of Compound A or matching placebo. Participants will fast for at least 10 hours prior to dosing, and participants will remain fasted until at least 4 hours after administration.

### Multiple Dose

On Day 5 through Day 10, subjects will receive Compound A BID or matching placebo. On Day 11, subjects will receive a morning dose (last dose) of Compound A or matching placebo.

On Days 5 and 11, participants will fast for at least 10 hours prior to dosing, and participants will remain fasted until at least 4 hours after administration of the morning dose.

In the mornings of Days 6−10, participants will also be dosed in the fasted state; however, approximately 2 hours after dosing on these days, they will receive breakfast.

In the evenings of Days 5−10, participants will receive a second oral dose of Compound A or placebo, 12 hours after the morning dose. The evening dose will be administered approximately 2 hours after start of dinner.

Study treatment will be administered with 240 mL of water.

### Example 4: A Multicenter, Randomized, Double-Blind, Placebo-Controlled, Phase 2 Study of the Efficacy, and the Safety and Tolerability of Compound a in Participants with Pulmonary Fibrosis

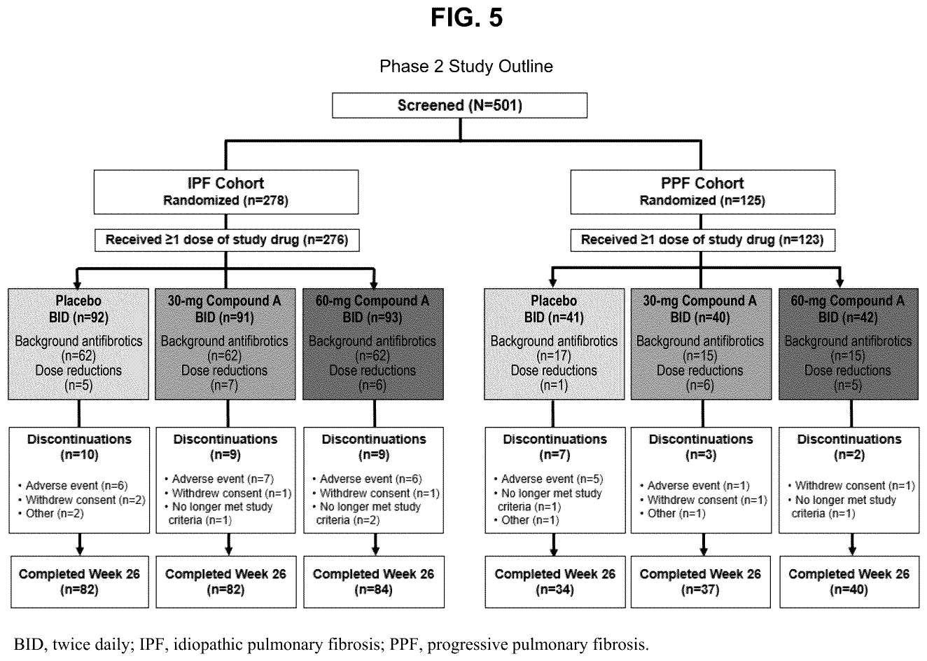

Safety and efficacy topline data from the final analysis of the IPF cohort in the Phase 2 Study support the continued development of Compound A in both IPF and PPF. A schematic showing the outline of both cohorts of the trial is shown in FIG. 5.

### Example 4A: IPF Cohort

A total of 278 participants with IPF were randomized and 276 participants were treated as of the final analyses data cut-off (4 Aug. 2022). Male and female participants 240 years of age with IPF and percent predicted forced vital capacity (ppFVC) 2240% and percent predicted DLCO (ppDLCO)≥25%; diagnosed within 7 years of screening; having a centrally-read chest HRCT obtained at screening that was consistent with UIP or probable UIP, or having a lung biopsy consistent with UIP, were eligible to enter the study. The mean baseline ppFVC for all subjects was 76.5%. In the primary IPF cohort, participants were randomized (1:1:1) to receive 30 mg or 60 mg Compound A or placebo (PBO) twice daily for 26 weeks. Participants were stratified by standard of care (SoC) IPF therapy (pirfenidone versus nintedanib versus none) and region (Japan versus Rest of World) at randomization.

The primary objective of the study was to determine the rate of change in ppFVC from baseline to Week 26. The primary objective was evaluated with a two estimand framework to handle the intercurrent event of dose reduction to 10 mg BID or matching PBO when pre-specified low BP criteria is met. The primary estimand is adopted to estimate treatment effect when dose reduction is implemented. The supplementary estimand is adopted to estimate the effect of treatment without dose reduction.

Of the 276 treated participants that contributed to these analyses, 90% (n=248) completed the 26-week treatment phase and 10% (n=28) were treatment discontinued. Treatment discontinuations due to TEAEs were evenly balanced across treatment groups (PBO: 9.8%; 30 mg: 9.9%; 60 mg: 6.5%).

Table 8 shows the baseline demographics and clinical characteristics of the subject population in the IPF cohort.

TABLE 8
Baseline Demographics and Clinical Characteristics

| | IPF Cohort | | | | | |
|---|---|---|---|---|---|---|
| | | | 30-mg | | 60-mg | |
| | Placebo | | Compound A | | Compound A | |
| Parameter* | (n = 92) | | (n = 91) | | (n = 93) | |
| Demographics | | | | | | |
| Age, years | 69.0 ± 6.7 | | 69.5 ± 7.3 | | 68.8 ± 7.9 | |
| Male sex, no. (%) | 76 | (82.6) | 77 | (84.6) | 69 | (74.2) |
| Race, no. (%) | | | | | | |
| White | 65 | (70.7) | 64 | (70.3) | 64 | (68.8) |
| Asian | 25 | (27.2) | 25 | (27.5) | 27 | (29.0) |
| Other | 2 | (2.2) | 2 | (2.2) | 2 | (2.2) |
| Clinical characteristics | | | | | | |
| Weight, kg | 76.5 ± 15.0 | | 76.5 ± 14.1 | | 75.8 ± 15.6 | |
| BMI, kg/m² | 27.2 ± 4.1 | | 26.9 ± 4.0 | | 27.1 ± 4.5 | |
| Cigarette smoking history, no. (%) | | | | | | |
| Never | 32 | (34.8) | 30 | (33.0) | 31 | (33.3) |
| Current | 1 | (1.1) | 1 | (1.1) | 2 | (2.2) |
| Former | 59 | (64.1) | 60 | (65.9) | 60 | (64.5) |
| Concurrent background treatment^{†}, no. (%) | | | | | | |
| Pirfenidone | 26 | (28.3) | 26 | (28.6) | 25 | (26.9) |
| Nintedanib | 36 | (39.1) | 36 | (39.6) | 37 | (39.8) |
| None | 30 | (32.6) | 29 | (31.9) | 31 | (33.3) |

| | | | |
|---|---|---|---|
| Time since diagnosis, years | 2.7 ± 1.7 | 2.8 ± 1.6 | 2.8 ± 1.7 |
| No. | 89 | 82 | 84 |
| FVC, mL | 2741 ± 810 | 2768 ± 770 | 2641 ± 768 |
| FVC, percent of predicted | 77 ± 19 | 77 ± 19 | 76 ± 17 |
| No. | 65 | 66 | 68 |
| | 51.2 ± 18.6 | 47.8 ± 14.3 | 48.4 ± 17.3 |
| DL_{CO}, | | | |
| percent of predicted, | | | |
| hemoglobin-corrected | | | |
| No. | 80 | 81 | 82 |
| 6MWT, meters | 389.4 ± 113.8 | 399.3 ± 102.1 | 401.8 ± 115.7 |
| No. | 89 | 88 | 90 |
| Quantitative lung fibrosis | 17.3 ± 11.0 | 17.3 ± 10.8 | 17.2 ± 10.4 |
| (HRCT), % | | | |

*Values before and after plus-minus symbols represent means ± SD.
^{†}Patients with PPF were permitted to remain on background antifibrotics ± ILD-targeted immunosuppressants (see Supplementary Table S1).
6MWT, 6-minute walk test; BMI, body mass index; DL_{CO}, diffusing capacity of the lung for carbon monoxide; FVC, forced vital capacity; HRCT, high-resolution computed tomography; ILD, interstitial lung disease; IPF, idiopathic pulmonary fibrosis; PPF, progressive pulmonary fibrosis; SD, standard deviation.

Summary results are described in Tables 9−12; and additional results from the final analysis from the IPF cohort are described below.

### Primary Estimand:

Analysis Strategy: The primary estimand was to evaluate the efficacy of Compound A at 30 mg or 60 mg twice daily, compared to PBO in IPF participants who met the enrollment criteria, with or without use of SoC. The rate of decline in ppFVC (%) from baseline to Week 26 was compared using the difference between each dose and PBO as a population level summary regardless of dose reduction or treatment discontinuation for any reason (treatment policy strategy).

### Efficacy Results Based on Primary Estimand:

In the overall population, the 60 mg dose group showed favorable treatment responses at Week 26, measured as rate of decline of ppFVC and decline of FVC (mL), compared to PBO under both primary estimand and the supplementary estimand frameworks (Table 8). The 30 mg dose group did not demonstrate efficacy (data not shown).

In the overall population, the 60 mg dose showed a treatment difference in the rate of decline in ppFVC of 1.45±0.81 (mean±standard error of the mean [SEM]) [95% CI−0.133, 3.028]. This corresponds to an overall relative reduction of 54%, compared to PBO slope of decline (−2.67±0.57).

When analyzing the rate of decline in FVC in mL (adjusted for age, gender, and height as measured by the slope difference of the 60 mg arm [−54.3±20.76] by the difference in slope of the PBO arm [−101.2±20.45]), the 60 mg dose corresponds to a 46.9 ml relative treatment difference compared to PBO (46.9±29.14 (mean±SEM) [95% CI−10.3, 104.1]).

Subgroup analyses of background SoC (SoC vs none) demonstrated favorable responses on the primary endpoint in the 60 mg group in contrast to the PBO group.

Of those on background SoC (68% of the overall population), the 60 mg dose showed a treatment difference versus PBO of 1.22±0.87 (mean±SEM) [95% CI−0.486, 2.929], which corresponds to a 39% relative treatment benefit compared to PBO.

Of those not on background SoC (32% of the overall population), the 60 mg dose showed a treatment difference versus PBO of 2.01±1.7 (mean±SEM) [95% CI−1.327, 5.355], which corresponds to a 113% relative treatment benefit compared to PBO.

### Supplementary Estimand:

Analysis Strategy: The supplementary estimand for the primary objective is to evaluate the efficacy of Compound A at 30 mg or 60 mg twice daily without dose reduction, compared to the PBO in IPF subjects who met the enrollment criteria, with or without use of SoC. The rate of decline in ppFVC (%) from baseline to Week 26 has been compared using the difference between each dose and PBO as a population level summary regardless of treatment discontinuation for any reason (treatment policy strategy). In the event of dose reduction, data collected after dose reduction will not be considered relevant to the treatment effect of interest and therefore, will be treated as missing (while-on-treatment strategy).

Dose reductions to Compound A 10 mg BID vs. matching PBO were implemented due to protocol-defined low BP criteria in 18 (6.5%) participants and approximately evenly distributed across arms (PBO: 5 (5.4%); 30 mg: 7 (7.7%); 60 mg: 6 (6.5%))

In the overall population, the 60 mg dose showed a treatment difference in the rate of decline in ppFVC of 1.77±0.82 (mean±SEM) [95% CI 0.162, 3.370]. This corresponds to an overall relative reduction of 62%, compared to PBO slope of decline (−2.84±0.57).

When analyzing FVC in mL (adjusted for age, gender, and height, as measured by the difference in slope of the 60 mg arm [−47.2±20.92] by the difference in slope of the PBO arm [−108.7±20.58]), the 60 mg dose corresponds to a 61.4 ml relative treatment difference compared to PBO.

Subgroup analyses of background SoC (SoC vs none) demonstrated favorable responses on the primary endpoint in the 60 mg group in contrast to the PBO group.

Of those on background SoC (68% of the overall population), the 60 mg dose showed a treatment difference versus PBO on the primary endpoint (rate of change of ppFVC) of 1.41±0.89 (mean±SEM) [95% CI−0.341, 3.151], which corresponds to a 44% relative treatment benefit compared to PBO.

Of those not on background SoC (32% of the overall population), the 60 mg dose showed a treatment difference versus PBO on the primary endpoint (rate of change of ppFVC) of 2.55±1.73 (mean±SEM) [95% CI−0.84, 5.948], which corresponds to a 123% relative treatment benefit compared to PBO.

Subgroup analyses of participants enrolled while on stable background SoC (defined as either nintedanib or pirfenidone) (68% of the overall population) and those enrolled without being on SoC (No SoC; 32% of the overall population), showed favorable efficacy with 60 mg compared to PBO (Table 8).

Subgroup analysis based on sex revealed differing rates of decline. Data not shown.

TABLE 9
Estimated Difference in Rate of Decline from Baseline to Week 26

| | Primary Estimand^{a} | | | | Supplementary Estimand^{b} | | | |
|---|---|---|---|---|---|---|---|---|
| | | Slope | | | | Slope | | |
| | | Difference | | | | Difference | | |
| ppFVC (%) | Population | (SE) | | 95% CI | Population | (SE) | | 95% CI |
| 60 mg -PBO | Overall | 1.45 | (0.81) | −0.133, 3.028 | Overall | 1.77 | (0.82) | 0.162, 3.37 |
| | SoC | 1.22 | (0.87) | −0.486, 2.929 | SoC | 1.41 | (0.89) | −0.341, 3.151 |
| | No SoC | 2.01 | (1.7) | −1.327, 5.355 | No Soc | 2.55 | (1.73) | −0.84, 5.948 |
| | | Slope | | | | Slope | | |
| | | Difference | | | | Difference | | |
| FVC (mL) | | (SE) | | 95% CI | | (SE) | | 95% CI |
| 60 mg-PBO | Overall | 46.9 | (29.14) | −10.3, 104.1 | Overall | 61.4 | (29.35) | 3.8, 119.0 |

CI = confidence interval; FVC = forced vital capacity; PBO = placebo; ppFVC = percent predicted forced vital capacity; SE = standard error; SoC = standard of care.
^{a}Under the primary estimand, all observations are used for the analysis regardless of dose reduction to 10 mg BID or matching PBO for meeting low BP criteria.
^{a}Under the supplementary all estimand, observations are used for analysis up to the time of dose reduction to 10 mg BID or matching PBO for meeting low BP criteria.

TABLE 10
Estimated Rate of Decline in ppFVC from Baseline to Week 26

| | Primary Estimand | | | Supplementary Estimand | | | |
|---|---|---|---|---|---|---|---|
| Group | Population | Slope (SE) | 95% C.I. | Population | Slope (SE) | | 95% C.I. |
| PBO | Overall | −2.67 (0.57) | −3.782, −1.565 | Overall | −2.84 | (0.57) | −3.968, −1.719 |
| | SoC | −3.12 (0.61) | −4.321, −1.91 | SoC | −3.18 | (0.62) | −4.403, −1.951 |
| | No SoC | −1.78 (1.18) | −4.093, 0.532 | No SoC | −2.083 | (1.21) | −4.454, 0.288 |
| 60 mg | Overall | −1.23 (0.57) | −2.353, −0.10 | Overall | −1.08 | (0.58) | −2.221, 0.067 |
| | SoC | −1.89 (0.62) | −3.103, −0.686 | SoC | −1.772 | (0.63) | −3.015, −0.529 |
| | No SoC | 0.23 (1.23) | −2.178, 2.646 | No SoC | 0.471 | (1.23) | −1.958, 2.899 |

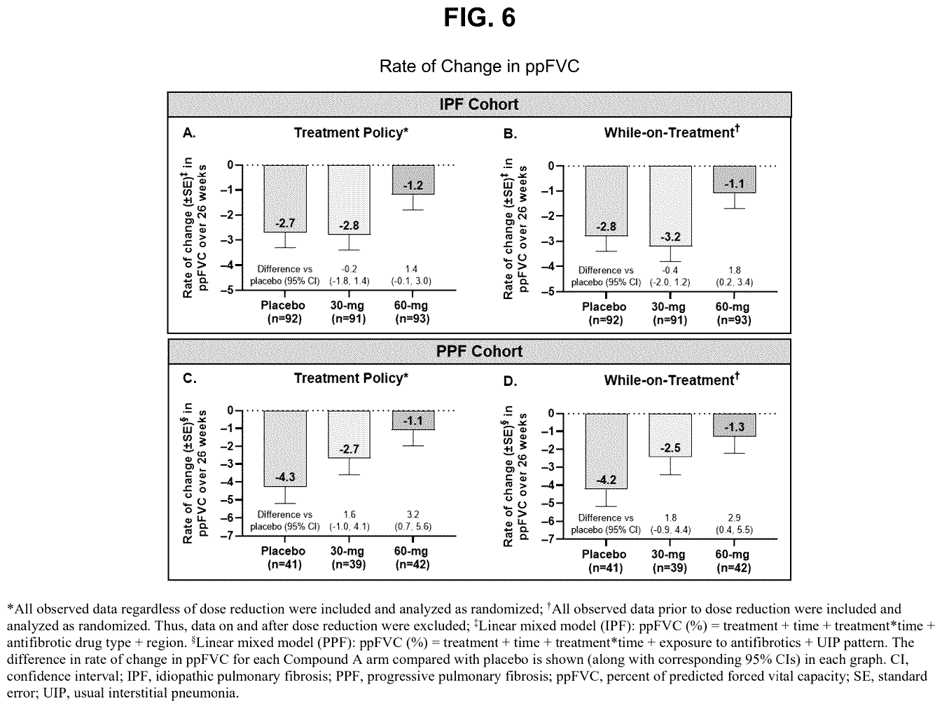

Additional data for the IPF cohort is shown in FIGS. 6-9. As shown in FIG. 6, in the treatment policy strategy, the rate of change in ppFVC over 26 weeks in patients with IPF was −2.7% for placebo vs −2.8% and −1.2% for the 30-mg and 60-mg Compound A arms, respectively. The treatment difference between the 60-mg arm vs placebo was 1.4% (95% CI, −0.1 to 3.0), a relative reduction of 54% (FIG. 6A). Using the while-on-treatment strategy, the rate of change in ppFVC was −2.8% for placebo vs −3.2% and −1.1% for the 30-mg and 60-mg Compound A arms, respectively. The treatment difference between the 60-mg Compound A arm vs placebo was 1.8% (95% CI, 0.2 to 3.4), a 62% relative reduction (FIG. 6B). In the Bayesian analysis, the posterior probability of a positive treatment difference for 60-mg Compound A vs placebo was >95% under both estimand strategies.

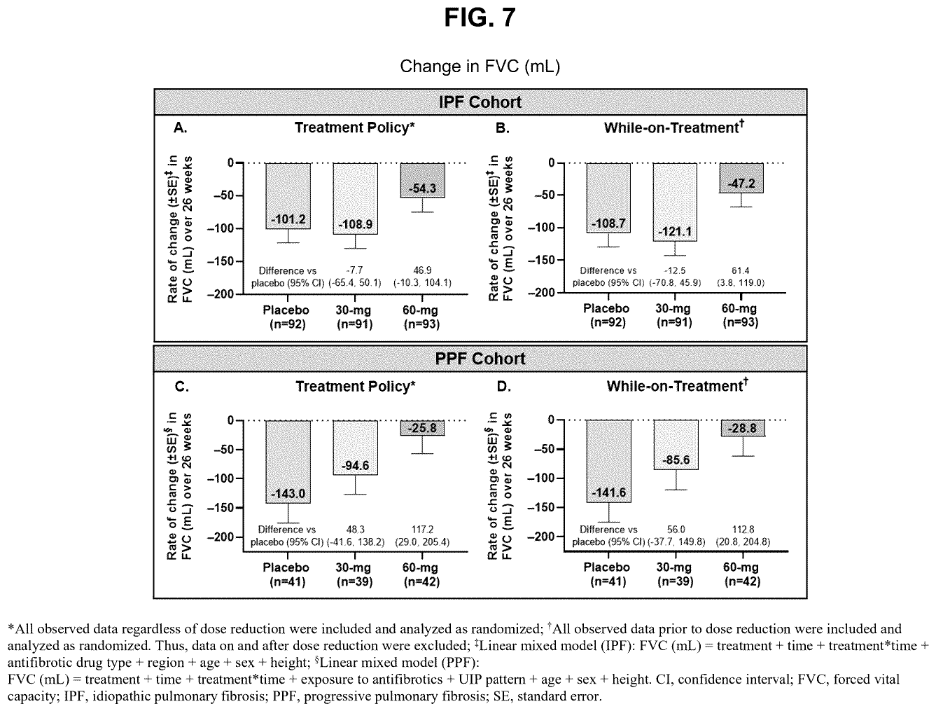

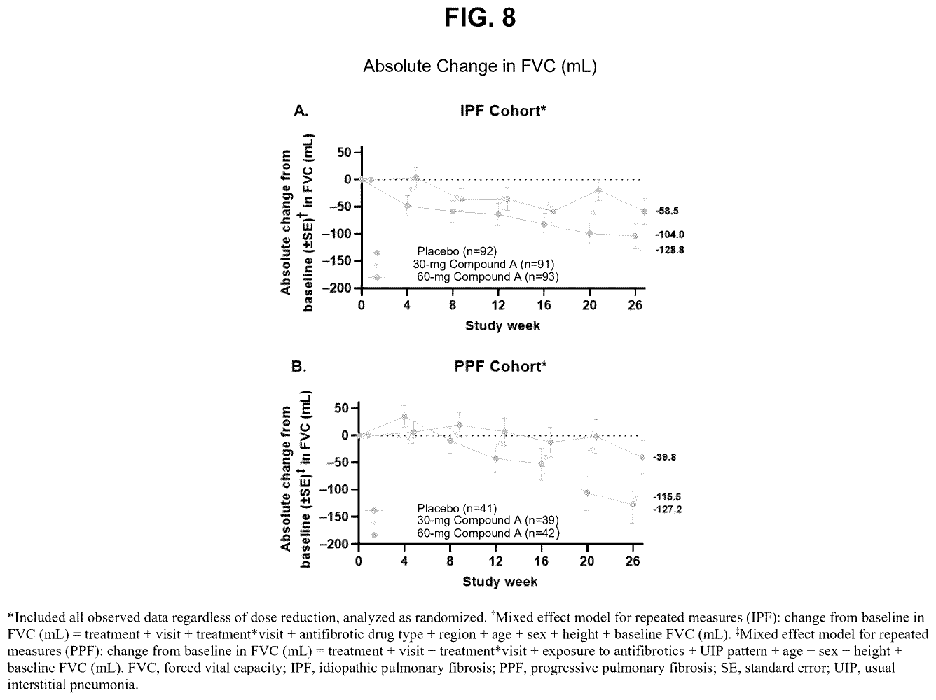

Rate of change in FVC (mL) for the IPF cohort is shown in FIG. 7 while FIG. 8 shows the absolute change in FVC. The adjusted mean treatment difference in absolute change in FVC (mL) between the 60-mg Compound A and placebo arms at week 26 was 45.5 mL (FIG. 8A).

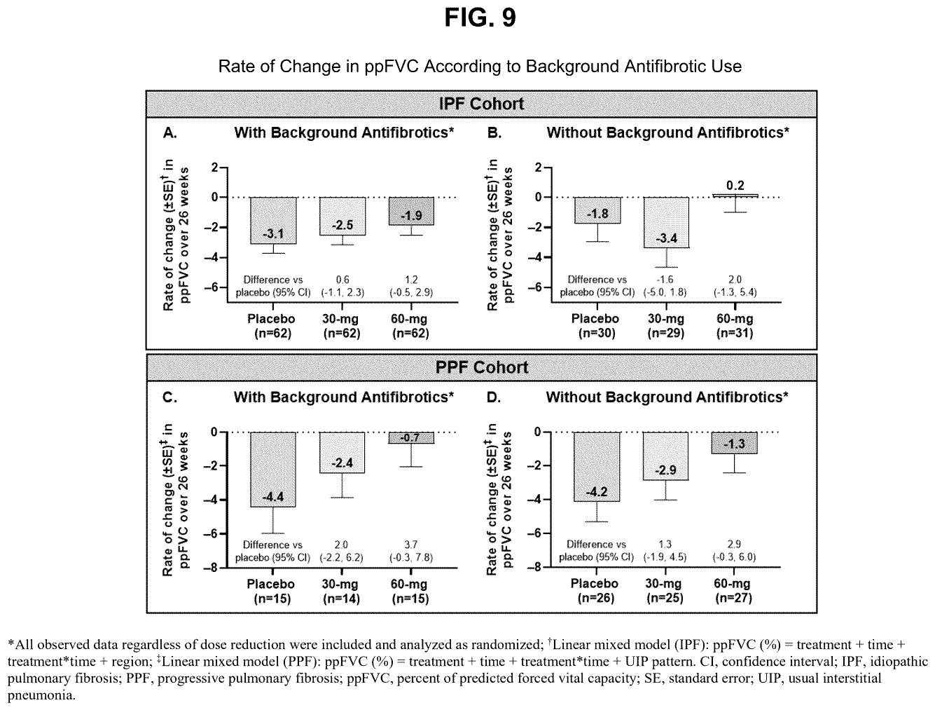

FIG. 9 shows the rate of change in ppFVC for subjects with and without background antifibrotic treatment in the IPF cohort. The treatment differences in rates of change in ppFVC were consistent between both groups (FIGS. 9A and 9B).

### Efficacy Results Summary:

The 30 mg dose group did not demonstrate efficacy compared to PBO in the overall population with and without SoC (data not shown).
The 60 mg dose group demonstrated favorable treatment responses versus PBO in the overall population with and without SoC, and for the sub-group of participants on background SoC as well as for those not on SoC.
These results indicate that Compound A has a favorable impact on FVC compared with PBO when used alone or on background therapy with nintedanib or pirfenidone.

In general, the overall adverse events among subjects with at least one TEAE were more frequently observed in the PBO group (Table 11). Discontinuations due to TEAE were evenly balanced across treatment groups (PBO: 9.8%; 30 mg: 9.9%; 60 mg: 6.5%).

TABLE 11
Summary of Select Safety Data

| | PBO | 30 mg | 60 mg | |
|---|---|---|---|---|
| Category, n (%) | (N = 92) | (N = 91) | (N = 93) | Active Total^{a} |
| Subjects with at least one TEAE | 74 (80.4) | 69 (75.8) | 69 (74.2) | 138 (75.0) |
| Subjects with at least one AESI | 19 (20.7) | 18 (19.0) | 9 (9.7) | 27 (14.7) |
| Subjects with at least one TESAE | 16 (17.4) | 10 (11.0) | 10 (10.8) | 20 (10.9) |
| Subjects discontinuing study | 9 (9.8) | 9 (9.9) | 6 (6.5) | 15 (8.2) |
| treatment due to a TEAE | | | | |
| Subjects with at least one | 20 (21.7) | 23 (25.3) | 25 (26.9) | 48 (26.1) |
| TEAE related to the study | | | | |
| treatment | | | | |
| Subjects who died due to a TEAE | 2 (2.2) | 3 (3.3) | 4 (4.3) | 7 (3.8) |
| Subjects who died | 4 (4.4) | 4 (4.4) | 5 (5.4) | 9 (4.9) |

AESI = adverse event of special interest; PBO = placebo; TEAE = treatment emergent adverse event; TESAE = treatment emergent serious adverse event.
^{a}Active total includes both 30 mg and 60 mg groups

### Safety Assessments:

In general, overall safety events were more prevalent in the PBO groups.
Treatment-emergent adverse event (TEAE):
   PBO: 74 (80.4%); 30 mg: 69 (75.8%); 60 mg: 69 (74.2%)
Adverse event of special interest (AESI):
   PBO: 19 (20.7%); 30 mg: 18 (19.8%); 60 mg: 9 (9.7%)
Treatment-emergent serious adverse event (TESAE):
   PBO: 16 (17.4%); 30 mg: 10 (11.0%); 60 mg: 10 (10.8%)
Discontinuations due to a TEAE:
   PBO: 9 (9.8%); 30 mg: 9 (9.9%); 60 mg: 6 (6.5%)
TEAE related to study treatment:
   PBO: 20 (21.7%); 30 mg: 23 (25.3%); 60 mg: 25 (26.9%)
No predominant system organ class or preferred term reported for treatment discontinuations
Total deaths were 13:
   PBO: 4 (4.4%); 30 mg: 4 (4.4%); 60 mg: 5 (5.4%).
Causes of death were: 9 due to disease progression; 3 due to pneumonia; 1 due to congestive heart failure.
TEAE-related deaths: 9 (3.3%) participants of which occurred during the study or within 28 days of the last dose of treatment.
PBO: 2 (2.2%); 30 mg: 3 (3.3%); 60 mg: 4 (4.3%).
An additional 4 participants died beyond the 28-day TEAE window.

Blood Pressure Monitoring: Based on the pre-specified low BP safety monitoring and criteria specified in the protocol, those on 60 mg had the lowest frequencies of orthostatic intolerance, orthostatic hypotension, or pre-specified symptomatic or asymptomatic low BP criteria.

In the overall population, the following orthostatic intolerance, orthostatic hypotension, or the symptomatic or asymptomatic low BP events were observed:
Orthostatic intolerance observed in 19 (6.9%) participants.
PBO: 7 (7.6%); 30 mg: 10 (11.0%); 60 mg: 2 (2.2%)
Orthostatic hypotension observed in asymptomatic low BP in 54 (19.6%) participants. Note that orthostatic hypotension is defined as a drop in SBP of ≥20 mmHg or DBP of ≥10 mmHg with an assumption of an upright posture from either supine or seated to upright position.
PBO: 19 (20.7%); 30 mg: 21 (23.1%); 60 mg: 14 (15.1%)
Orthostatic hypotension observed in symptomatic low BP in 21 (7.5%) participants.
PBO: 7 (7.6%); 30 mg: 9 (9.9%), 60 mg: 5 (5.4%)
Asymptomatic low blood pressure observed in 59 (21.4%) of participants.
PBO: 20 (21.7%); 30 mg: 22 (24.2%); 60 mg: 17 (18.3%)
Symptomatic low blood pressure observed in 27 (9.8%) participants.
PBO: 10 (10.9%); 30 mg: 11 (12.1%); 60 mg: 6 (6.5%)
Dose reductions to Compound A 10 mg BID vs. matching PBO were implemented due to protocol-defined low BP criteria (shown below) in 18 (6.5%) participants and approximately evenly distributed across arms: PBO: 5 (5.4%); 30 mg: 7 (7.7%); 60 mg: 6 (6.5%)

### Asymptomatic Blood Pressure Reduction Criteria

Patient experiences any of the following, confirmed by retest within 15 minutes:
Seated systolic blood pressure <85 mmHg
Seated diastolic blood pressure <55 mmHg
Orthostatic hypotension

### Symptomatic Blood Pressure Reduction Criteria

Patient experiences symptoms that, in the investigator's opinion, could be associated with a reduction in blood pressure and also experiences at least one of the following, confirmed by retest within 15 minutes:
Seated systolic blood pressure <100 mmHg or seated diastolic blood pressure <60 mmHg
Decrease of seated systolic blood pressure of ≥20 mmHg from the previous visit or decrease of seated diastolic blood pressure of ≥10 mmHg from the previous visit
Orthostatic hypotension
Orthostatic tachycardia

Based on the pre-specified BP monitoring and low BP criteria in the protocol, these data demonstrate that the dose of Compound A 60 mg twice daily (BID), as compared to PBO, was not associated with any increased risk of orthostatic intolerance, orthostatic hypotension, orthostatic tachycardia, or symptomatic or asymptomatic low BP (Table 12). However, postdose reductions in SBP were observed with the 30 mg and 60 mg on Day 1 of dosing. The nadir post-dose mean reductions from baseline in sitting SBP were noted at 2 hours post-dose (PBO: −2.1 mmHg; Compound A 30 mg: −10.5 mmHg; Compound A 60 mg: −14.1 mmHg). These reductions were not associated with any clinical sequelae and were self-limited in nature.

TABLE 12
Summary of Low Blood Pressure Data

| Category, n (%) | PBO (N = 92 | 30 mg (N = 91) | 60 mg (N = 93) | Total |
|---|---|---|---|---|
| Orthostatic | 7 (7.6) | 10 (11.0) | 2 (2.2) | 19 (6.9) |
| Intolerance | | | | |
| Asymptomatic low | 20 (21.7) | 22 (24.2) | 17 (18.3) | 59 (21.4) |
| blood pressure | | | | |
| Orthostatic | 19 (20.7) | 21 (23.1) | 14 (15.1) | 54 (19.6) |
| hypotension* | | | | |
| Symptomatic low | 10 (10.9) | 11 (12.1) | 6 (6.5) | 27 (9.8) |
| blood pressure | | | | |
| Orthostatic | 7 (7.6) | 9 (9.9) | 5 (5.4) | 21 (7.6) |
| hypotension* | | | | |

PBO = placebo
*Orthostatic hypotension is defined as a drop in SBP of ≥20 mmHg or DBP of ≥10 mmHg with an assumption of an upright posture from either supine or seated to upright position.

The number of dose reductions (and percent of the total subjects in the group) in the IPF cohort were: 5 (5.4) for placebo; 7 (7.7) for 30 mg Compound A; and 6 (6.5) for 60 mg of Compound A.

The number of dose reductions (and percent of the total subjects in the group) in the IPF cohort were: 5 (5.4) for placebo; 7 (7.7) for 30 mg Compound A; and 6 (6.5) for 60 mg of Compound A.

### Example 4B: PPF Cohort

A total of 123 participants with PPF were randomized. Participants (age ≥21 years) were randomized (1:1:1) to receive 30 mg or 60 mg Compound A or PBO twice daily for 26 weeks. Subjects with a centrally read HRCT obtained at screening demonstrating evidence of ≥10% parenchymal fibrosis within the whole lung along with:
(a) evidence of ILD progression within the 24 months before screening, defined as either: a decline in the relative ppFVC of ≥10%, or a decline in the relative ppFVC of ≥5% to <10% along with increased extent of fibrosis on pre-screening thoracic computed tomography compared with prior imaging, or symptoms associated with progression of ILD along with an increased extent of fibrosis on pre-screening thoracic computed tomography compared with prior imaging;
(b) interstitial lung diseases of diverse etiologies, excluding connective tissue disease-associated interstitial lung disease (except rheumatoid arthritis-associated interstitial lung disease) and sarcoid; were enrolled. Immunosuppressive medications (mycophenolate mofetil, mycophenolic acid, azathioprine and/or tacrolimus) were permitted only if dosing is stable for ≥6 months prior to screening. If receiving antifibrotic agents pirfenidone or nintedanib, patients had to receive a stable dosage for ≥3 months prior to screening and during the screening period; if not receiving pirfenidone or nintedanib, patients had to be naive to both drugs or not have received either 4 weeks prior to Day 1. A total of 47 (38.2%) subjects were receiving antifibrotic treatment (with or without immunosuppression therapy). Of those subjects, 34 were being treated with nintedanib and 13 with pirfenidone. Participants were stratified by usual interstitial pneumonia (UIP) pattern (presence versus absence) and background therapy (antifibrotics+/−ILD-targeted immunosuppression versus ILD-targeted immunosuppression alone versus none). UIP pattern was present in 52% of subjects and unclassifiable ILD was the most common disease categorization.

The study was not powered to detect statistical significance (no formal testing). No target effect size was defined in the study design. There were two estimand approaches for analysis:
Treatment Policy: Effect of treatment with dose reduction as part of treatment regimen.

While-On Treatment: Effect of treatment without dose reduction.

Of the 123 treated participants that contributed to these analyses, 90.2% (n=111) completed the 26-week treatment phase and 9.8% (n=12) were treatment discontinued (6 (4.9%) were discontinued for adverse events). Treatment discontinuations across treatment groups (PBO: 17.1%; 30 mg: 7.5%; 60 mg: 4.8%). A total of 91 (74%) subjects continued into a 26 Week Optional Treatment Extension (OTE).

The main efficacy endpoint, rate of FVC decline (in % of predicted) from baseline to wk-26 in the PPF cohort, was estimated using a linear mixed-effects model utilizing all FVC timepoints. It was analyzed under a primary estimand (utilizing all data, treatment policy strategy), and under supplementary estimand (utilizing all data up to the point of dose reduction, while-on-treatment strategy). Analysis was performed in the overall population as well as in sub-groups by 1) background anti-fibrotic treatment or not 2) UIP radiographic pattern (presence or not). The mean baseline ppFVC was 66.7% for the PPF cohort.

Table 13 shows the baseline demographics and clinical characteristics of subjects in the PPF cohort while Table 14 shows their baseline disease characteristics.

TABLE 13
Baseline Demographics and Clinical Characteristics

| | PPF Cohort | | | | | |
|---|---|---|---|---|---|---|
| | | | 30-mg | | 60-mg | |
| | Placebo | | Compound A | | Compound A | |
| Parameter* | (n = 41) | | (n = 40) | | (n = 42) | |
| Demographics | | | | | | |
| Age, years | 68.8 ± 8.1 | | 71.4 ± 7.9 | | 67.9 ± 8.4 | |
| Male sex, no. (%) | 20 | (48.8) | 23 | (57.5) | 22 | (52.4) |
| Race, no. (%) | | | | | | |
| White | 31 | (75.6) | 27 | (67.5) | 32 | (76.2) |
| Asian | 8 | (19.5) | 9 | (22.5) | 6 | (14.3) |
| Other | 2 | (4.9) | 4 | (10.0) | 4 | (9.5) |
| Clinical characteristics | | | | | | |
| Weight, kg | 71.9 ± 16.2 | | 73.5 ± 17.3 | | 74.9 ± 19.4 | |
| BMI, kg/m² | 26.9 ± 4.7 | | 26.3 ± 5.0 | | 27.0 ± 4.8 | |
| Cigarette smoking history, no. (%) | | | | | | |
| Never | 19 | (46.3) | 19 | (47.5) | 24 | (57.1) |
| Current | 0 | | 0 | | 0 | |
| Former | 22 | (53.7) | 21 | (52.5) | 18 | (42.9) |
| Concurrent background treatment^{†}, no. (%) | | | | | | |
| Pirfenidone | 3 | (7.3) | 4 | (10.0) | 6 | (14.3) |
| Nintedanib | 14 | (34.1) | 11 | (27.5) | 9 | (21.4) |
| None | 20 | (48.8) | 22 | (55.0) | 24 | (57.1) |

| | | | |
|---|---|---|---|
| Time since diagnosis, years | 2.8 ± 2.6 | 3.1 ± 2.7 | 3.9 ± 2.7 |
| No. | 39 | 36 | 39 |
| FVC, mL | 2126 ± 616 | 2246 ± 728 | 2113 ± 723 |
| FVC, percent of predicted | 68 ± 18 | 67 ± 16 | 65 ± 17 |
| No. | 26 | 30 | 32 |
| DL_{CO}, | 43.4 ± 13.0 | 45.7 ± 14.6 | 46.4 ± 12.9 |
| percent of predicted, | | | |
| hemoglobin-corrected | | | |
| No. | 36 | 36 | 38 |
| 6MWT, meters | 344.8 ± 109.6 | 345.3 ± 109.1 | 352.9 ± 103.7 |
| No. | 39 | 39 | 42 |
| Quantitative lung fibrosis | 18.1 ± 11.1 | 19.1 ± 11.9 | 23.9 ± 12.4 |
| (HRCT), % | | | |

*Values before and after plus-minus symbols represent means ± SD.
^{†}Patients with PPF were permitted to remain on background antifibrotics ± ILD-targeted immunosuppressants (see Supplementary Table S1).
6MWT, 6-minute walk test; BMI, body mass index; DL_{CO}, diffusing capacity of the lung for carbon monoxide; FVC, forced vital capacity; HRCT, high-resolution computed tomography; ILD, interstitial lung disease; IPF, idiopathic pulmonary fibrosis; PPF, progressive pulmonary fibrosis; SD, standard deviation.

TABLE 14
Baseline Disease Characteristics for the PPF Cohort

| | | 30-mg | 60-mg |
|---|---|---|---|
| | Placebo | Compound A | Compound A |
| Parameter, no. (%) | (n = 41) | (n = 40) | (n = 42) |
| UIP pattern present | 21 (51.2) | 22 (55.0) | 21 (50.0) |
| Disease classification | | | |
| Unclassifiable ILD | 11 (26.8) | 13 (32.5) | 9 (21.4) |
| Rheumatoid arthritis ILD | 10 (24.4) | 4 (10.0) | 6 (14.3) |
| Chronic hypersensitivity | 8 (19.5) | 6 (15.0) | 7 (16.7) |
| pneumonitis | | | |
| Idiopathic fibrotic NSIP | 3 (7.3) | 7 (17.5) | 6 (14.3) |
| Interstitial pneumonia with | 4 (9.8) | 2 (5.0) | 6 (14.3) |
| autoimmune features | | | |
| Other progressive ILD | 5 (12.2) | 8 (20.0) | 8 (19.0) |
| Concurrent background treatment | | | |
| Antifibrotics without ILD- | 15 (36.6) | 13 (32.5) | 13 (31.0) |
| targeted | | | |
| immunosuppressants | | | |
| ILD-targeted | 4 (9.8) | 3 (7.5) | 3 (7.1) |
| immunosuppressants | | | |
| without antifibrotics | | | |
| Antifibrotic + ILD- | 2 (4.9) | 2 (5.0) | 2 (4.8) |
| targeted | | | |
| immunosuppressants | | | |
| No background treatment | 20 (48.8) | 22 (55.0) | 24 (57.1) |

ILD, interstitial lung disease; NSIP, non-specific interstitial pneumonia; PPF, progressive pulmonary fibrosis; UIP, usual interstitial pneumonia.

Summary results are described in Tables 15−26; and additional results from the final analysis from the PPF cohort are described below.

TABLE 15
Estimated Difference in Rate of Decline in ppFVC
from Baseline to Week 26 in Different Subgroups

| | Treatment | N | | |
|---|---|---|---|---|
| | difference 60 | (60 mg/ | | Relative |
| Population | mg v placebo | placebo) | 95% CI | Reduction |
| Overall | 2.94 | 42/41 | 0.35 to 5.53 | 69% |
| UIP subgroup | 2.12 | 21/21 | −1.06 to 5.31 | 54% |
| Non UIP subgroup | 3.80 | 21/20 | −0.48 to 8.09 | 84% |
| Anti-fibrotic subgroup | 3.28 | 15/15 | −1.1 to 7.66 | 75% |
| No antifibrotic subgroup | 2.61 | 27/26 | −0.6 to 5.82 | 63% |

Efficacy of Compound A (60 mg) was shown in a population in which 37% of patients were on background antifibrotics.
60 mg relative reduction in PPF cohort (69%) was similar to IPF cohort (62%)
Clear dose response
Efficacy consistent in all sub-groups, i.e., irrespective of background antifibrotics or of UIP pattern.

TABLE 16
Main Analysis in FVC (% Predicted): Rate of Decline
in Overall Population - Primary Estimand - Treatment
Policy Strategy (Relative Reduction = 74%)

| | | | Difference | |
|---|---|---|---|---|
| Treatment | Slope (S.E.) | Difference | (S.E.) | 95% C.I. |
| Placebo | −4.27 (0.93) | | | |
| (n = 41) | | | | |
| 30 mg | −2.7 (0.9) | 30 mg - | 1.57 (1.29) | [−0.97, 4.11] |
| (n = 39) | | placebo | | |
| 60 mg | −1.11 (0.86) | 60 mg - | 3.15 (1.27) | [0.66, 5.64] |
| (n = 42) | | placebo | | |

TABLE 17
Main Analysis in FVC (% Predicted): Rate of Decline
in Overall Population - Supplementary Estimand -
While On Treatment (Relative Reduction = 69.3%)

| | | | Difference | |
|---|---|---|---|---|
| Treatment | Slope (S.E.) | Difference | (S.E.) | 95% C.I. |
| Placebo | −4.24 (0.94) | | | |
| (n = 41) | | | | |
| 30 mg | −2.45 (0.96) | 30 mg - | 1.78 (1.34) | [−0.86, 4.43] |
| (n = 39) | | placebo | | |
| 60 mg | −1.3 (0.92) | 60 mg - | 2.94 (1.32) | [0.35, 5.53] |
| (n = 42) | | placebo | | |

Additional data for the PPF cohort is shown in FIGS. 6-16. As shown in FIG. 6, the rate of change in ppFVC over 26 weeks in patients with PPF was −4.3% for placebo vs −2.7% and −1.1% for the 30-mg and 60-mg Compound A arms, respectively, per the treatment policy strategy. The treatment difference for 60-mg Compound A vs placebo was 3.2% (95% CI, 0.7 to 5.6), a relative reduction of 74% (FIG. 6C). Using the while-on-treatment strategy, the rate of change in ppFVC was −4.2% for placebo vs −2.5% and −1.3% for the 30-mg and 60-mg arms, respectively. The treatment difference between 60-mg Compound A vs placebo was 2.9% (95% CI, 0.4 to 5.5), a relative reduction of 69% (FIG. 6D).

Rate of change in FVC (mL) for the PPF cohort is shown in FIG. 7 while FIG. 8 shows the absolute change in FVC. The adjusted mean treatment differences in absolute change in FVC (mL) between the 60-mg Compound A and placebo arms at week 26 was 87.4 mL (FIG. 8B).

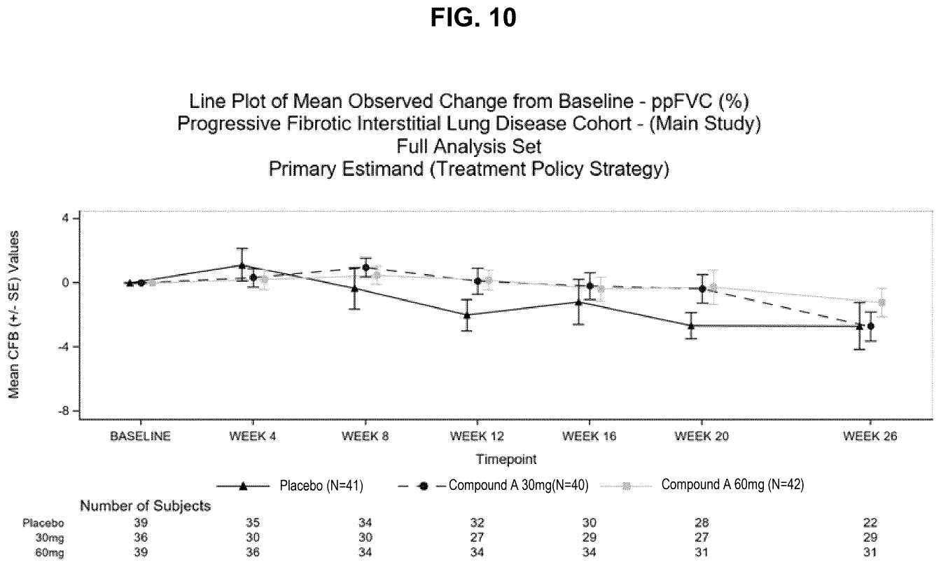

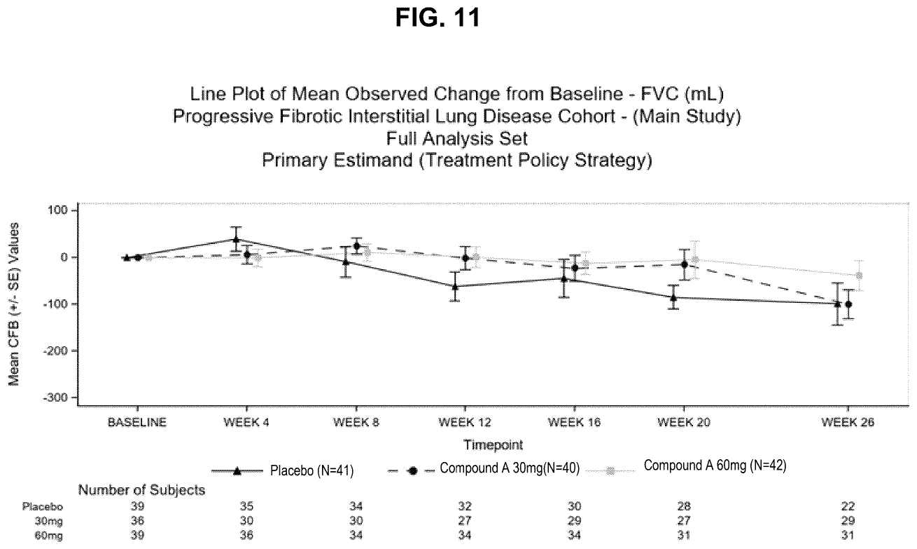

FIGS. 10 and 11 show the mean observed change from baseline to week 26 in ppFVC percentage and FVC in the PPF cohort. As shown in the figures, both 30 mg and 60 mg show improvement over placebo at week 8, but 30 mg shows more significant improvement after week 20 while 60 mg maintains the improvement over placebo through the end of week 26.

TABLE 18
Subgroup Analysis in FVC (% Predicted): Rate of Decline
in UIP Present Subjects - Primary Estimand - Treatment
Policy Strategy (Relative Reduction = 63%)

| | | | Difference | |
|---|---|---|---|---|
| Treatment | Slope (S.E.) | Difference | (S.E.) | 95% C.I. |
| Placebo | −3.95 (1.18) | | | |
| (n = 21) | | | | |
| 30 mg | −3.07 (1.09) | 30 mg - | 0.88 (1.6) | [−2.28, 4.04] |
| (n = 22) | | placebo | | |
| 60 mg | −1.46 (1.1) | 60 mg - | 2.49 (1.61) | [−0.68, 5.67] |
| (n = 21) | | placebo | | |

TABLE 19
Subgroup Analysis in FVC (% Predicted): Rate of Decline
in UIP Absent Subjects - Primary Estimand - Treatment
Policy Strategy (Relative Reduction = 83.6%)

| | | | Difference | |
|---|---|---|---|---|
| Treatment | Slope (S.E.) | Difference | (S.E.) | 95% C.I. |
| Placebo | −4.56 (1.48) | | | |
| (n = 20) | | | | |
| 30 mg | −2.31 (1.53) | 30 mg - | 2.25 (2.13) | [−1.95, 6.44] |
| (n = 17) | | placebo | | |
| 60 mg | −0.75 (1.38) | 60 mg - | 3.81 (2.02) | [−0.18, 7.79] |
| (n = 21) | | placebo | | |

TABLE 20
Subgroup Analysis in FVC (% Predicted): Rate of Decline
in UIP Present Subjects - Supplementary Estimand -
While on Treatment (Relative Reduction = 54%)

| | | | Difference | |
|---|---|---|---|---|
| Treatment | Slope (S.E.) | Difference | (S.E.) | 95% C.I. |
| Placebo | −3.94 (1.16) | | | |
| (n = 21) | | | | |
| 30 mg | −2.66 (1.12) | 30 mg - | 1.28 (1.61) | [−1.89, 4.46] |
| (n = 22) | | placebo | | |
| 60 mg | −1.82 (1.12) | 60 mg - | 2.12 (1.62) | [−1.06, 5.31] |
| (n = 21) | | placebo | | |

TABLE 21
Subgroup Analysis in FVC (% Predicted): Rate of Decline
in UIP Absent Subjects - Supplementary Estimand -
While on Treatment (Relative Reduction = 84.4%)

| | | | Difference | |
|---|---|---|---|---|
| Treatment | Slope (S.E.) | Difference | (S.E.) | 95% C.I. |
| Placebo | −4.5 (1.55) | | | |
| (n = 20) | | | | |
| 30 mg | −2.24 (1.68) | 30 mg - | 2.26 (2.28) | [−2.24, 6.77] |
| (n = 17) | | placebo | | |
| 60 mg | −0.7 (1.53) | 60 mg - | 3.8 (2.17) | [−0.48, 8.09] |
| (n = 21) | | placebo | | |

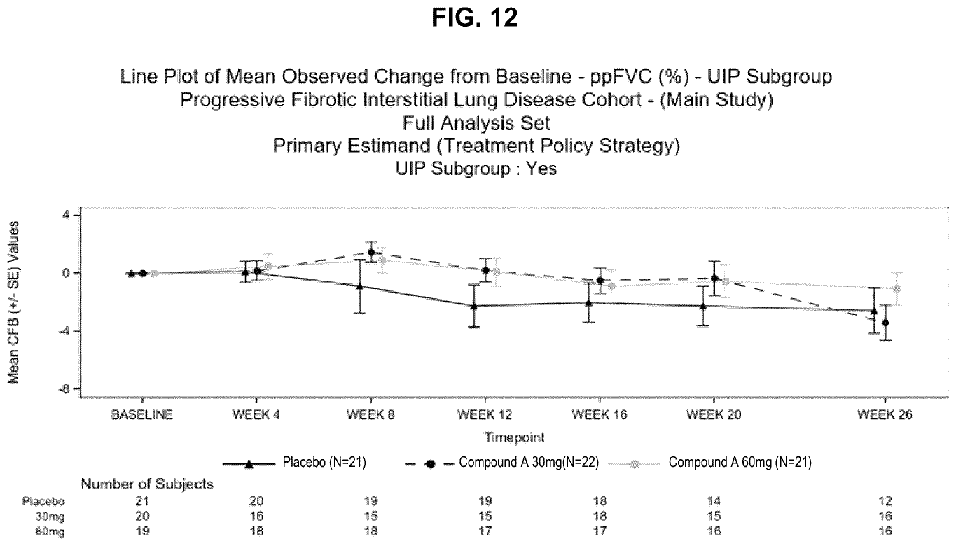

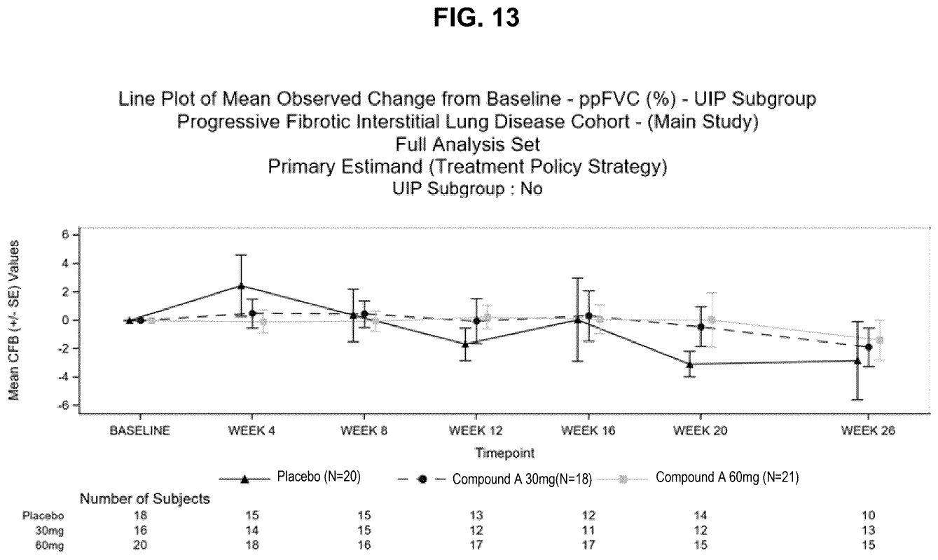

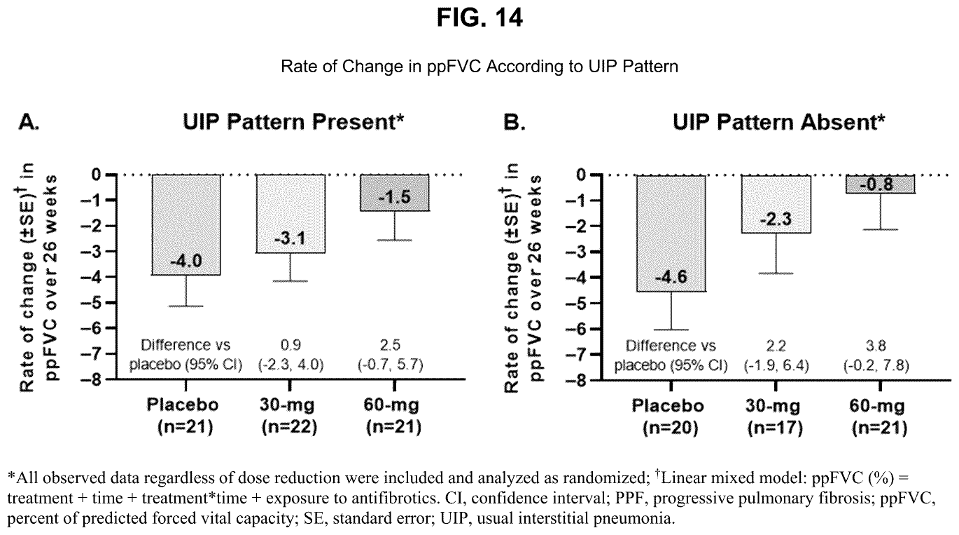

FIGS. 12 and 13 show the mean observed change from baseline to week 26 in ppFVC percentage and FVC in patients with and without UIP within the PPF cohort while FIG. 14 shows the rate of change in ppFVC in PPF patients with and without UIP. As shown in the figures, treatment differences were seen in the PPF cohort independent of UIP pattern presence or absence. Acute exacerbations of lung fibrosis were observed in 6 patients (2%) with IPF (placebo: n=2; 30-mg: n=3; 60-mg: n=1) and in 3 patients (2%) with PPF, all in the placebo arm.

TABLE 22
Subgroup Analysis in FVC (% Predicted): Rate of Decline
in Subjects Receiving Antifibrotic Treatment - Primary
Estimand - Treatment Policy (Relative Reduction = 85%)

| | | | Difference | |
|---|---|---|---|---|
| Treatment | Slope (S.E.) | Difference | (S.E.) | 95% C.I. |
| Placebo | −4.42 (1.55) | | | |
| (n = 15) | | | | |
| 30 mg | −2.43 (1.44) | 30 mg - | 1.99 (2.12) | [−2.19, 6.18] |
| (n = 14) | | placebo | | |
| 60 mg | −0.68 (1.37) | 60 mg - | 3.74 (2.1) | [−0.35, 7.83] |
| (n = 15) | | placebo | | |

TABLE 23
Subgroup Analysis in FVC (% Predicted): Rate of Decline in
Subjects Not Receiving Antifibrotic Treatment - Primary
Estimand - Treatment Policy (Relative Reduction = 68.5%)

| | | | Difference | |
|---|---|---|---|---|
| Treatment | Slope (S.E.) | Difference | (S.E.) | 95% C.I. |
| Placebo | −4.16 (1.15) | | | |
| (n = 26) | | | | |
| 30 mg | −2.88 (1.15) | 30 mg - | 1.28 (1.63) | [−1.93, 4.5] |
| (n = 25) | | placebo | | |
| 60 mg | −1.31 (1.11) | 60 mg - | 2.85 (1.6) | [−0.3, 6.01] |
| (n = 27) | | placebo | | |

TABLE 24
Subgroup Analysis in FVC (% Predicted): Rate of Decline in
Subjects Receiving Antifibrotic Treatment - Supplementary
Estimand - While on Treatment (Relative Reduction = 74.9%)

| | | | Difference | |
|---|---|---|---|---|
| Treatment | Slope (S.E.) | Difference | (S.E.) | 95% C.I. |
| Placebo | −4.38 (1.61) | | | |
| (n = 15) | | | | |
| 30 mg | −1.8 (1.58) | 30 mg - | 2.58 (2.25) | [−1.87, 7.03] |
| (n = 14) | | placebo | | |
| 60 mg | −1.1 (1.53) | 60 mg - | 3.28 (2.21) | [−1.1, 7.66] |
| (n = 15) | | placebo | | |

TABLE 25
Subgroup Analysis in FVC (% Predicted): Rate of Decline in
Subjects Not Receiving Antifibrotic Treatment - Supplementary
Estimand - While on Treatment (Relative Reduction = 62.6%)

| | | | Difference | |
|---|---|---|---|---|
| Treatment | Slope (S.E.) | Difference | (S.E.) | 95% C.I. |
| Placebo | −4.17 (1.16) | | | |
| (n = 26) | | | | |
| 30 mg | −2.83 (1.2) | 30 mg - | 1.34 (1.66) | [−1.94, 4.61] |
| (n = 25) | | placebo | | |
| 60 mg | −1.56 (1.15) | 60 mg - | 2.61 (1.63) | [−0.6, 5.82] |
| (n = 27T) | | placebo | | |

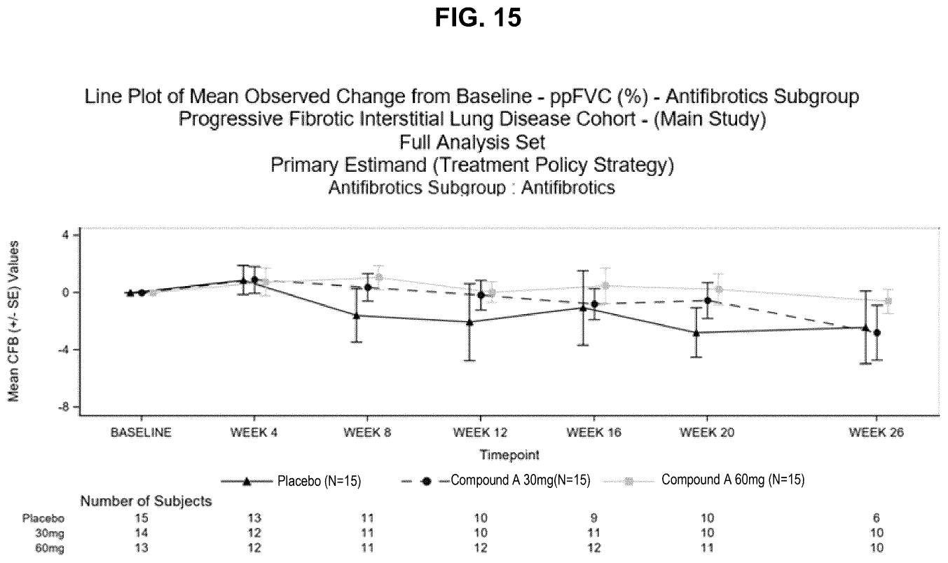

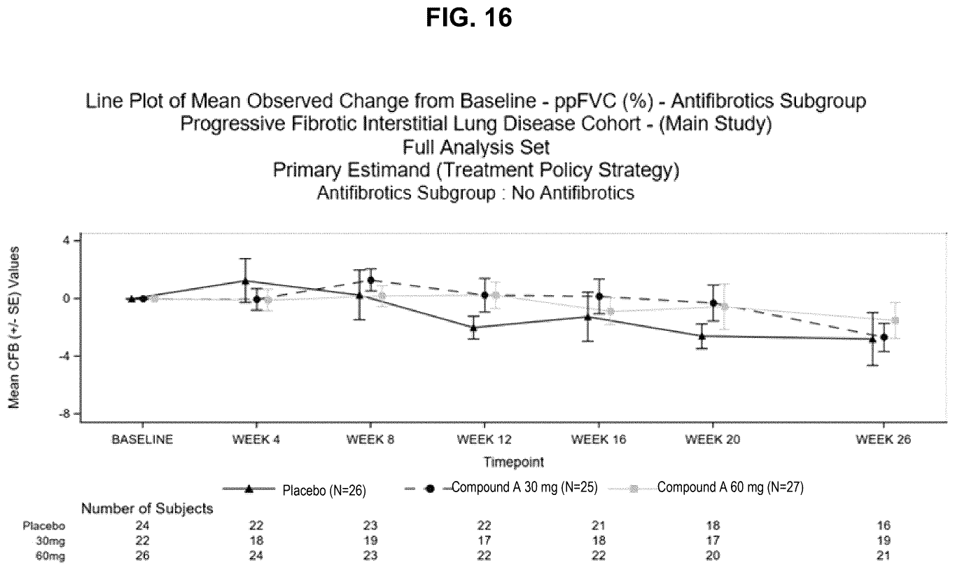

FIGS. 15 and 16 show the mean observed change from baseline to week 26 in ppFVC percentage and FVC in patients with and without additional antifibrotic treatment within the PPF cohort, while FIG. 9 shows the rate of change in ppFVC for subjects with and without background antifibrotic treatment in the PPF cohort. The treatment differences in rates of change in ppFVC were consistent with or without background antifibrotic use PPF cohorts (FIGS. 9C and 9D).

TABLE 26
Summary of Select Safety Data

| | | 30 mg | 60 mg |
|---|---|---|---|
| | Placebo | Compound A | Compound A |
| Patients, no. (%) | (n = 41) | (n = 40) | (n = 42) |
| ≥1 TEAE | 32 (78.0) | 33 (82.5) | 28 (66.7) |
| ≥1 TEAE related to study | 7 (17.1) | 7 (17.5) | 11 (26.2) |
| drug | | | |
| ≥1 TESAE | 13 (31.7) | 4 (10.0) | 5 (11.9) |
| Discontinuations due to | 6 (14.6) | 1 (2.5) | 0 |
| TEAEs | | | |
| Deaths due to TEAEs | 3 (7.3) | 0 | 0 |
| Diarrhea | 6 (14.6) | 6 (15.0) | 3 (7.1) |
| Cough | 4 (9.8) | 3 (7.5) | 5 (11.9) |
| COVID-19 | 2 (4.9) | 6 (15.0) | 6 (14.3) |
| Orthostatic hypotension | 1 (2.4) | 2 (5.0) | 4 (9.5) |
| Dyspnea | 6 (14.6) | 2 (5.0) | 0 |
| Nausea | 3 (7.3) | 4 (10.0) | 1 (2.4) |
| Dizziness | 4 (9.8) | 0 | 4 (9.5) |
| Hypotension | 0 | 4 (10.0) | 3 (7.1) |
| Hypertension | 0 | 0 | 1 (2.4) |
| Vomiting | 1 (2.4) | 3 (7.5) | 0 |
| Rash | 0 | 3 (7.5) | 0 |

COVID-19, coronavirus disease of 2019; IPF, idiopathic pulmonary fibrosis; PPF, progressive pulmonary fibrosis; TEAE, treatment-emergent adverse event; TESAE, treatment-emergent serious adverse event.

### Summary of Adverse Events

Subjects with at least one treatment emergent adverse event (TEAE): PBO-24%, 30 mg-10%, 60 mg-24%
Subjects with at least one treatment emergent serious adverse event (TESAE): PBO-32%, 30 mg-10%, 60 mg-12%
Subjects with at least one TEAE due to investigative medicinal product (IMP): PBO-17%, 30 mg-18%, 60 mg-26%
Seven subjects had an adverse ending leading to treatment discontinuation
   6 were in the PBO group and 1 was in the 30 mg dose group (due to hypotension)
Three subjects died due to a TEAE; all 3 were in the PBO group
   1 infectious pneumonia, 1 PE, 1 respiratory failure
Dose dependent BP findings reaffirm low BP safety risk with Compound A
   Day 1 post-dose reductions in blood pressure; without clinical consequences
   Dose dependent low BP thresholds (“events”): PBO-24%, 30 mg-30%, 60 mg-41%
   Dose reductions: PBO-2%, 30 mg-15%, 60 mg-12%
   AESI due to low BP: PBO-24%, 30 mg-10%, 60 mg-24%
No notable imbalances in marked laboratory abnormalities among treatment groups
No hepatobiliary toxicity

In patients with PPF, the nadir postdose reductions from baseline on day 1 in mean seated systolic blood pressure were −4.2 mmHg, −10.7 mmHg, and −12.7 mmHg for the placebo, 30 mg, and 60 mg Compound A arms, respectively. Prespecified blood pressure reduction criteria (shown below) increased in a dose-dependent manner across PPF cohort arms (Table 27).

### Asymptomatic Blood Pressure Reduction Criteria

Patient is experiencing any of the following, confirmed by retest within 15 minutes:
Seated systolic blood pressure <85 mmHg
Seated diastolic blood pressure <55 mmHg
Orthostatic hypotension

### Symptomatic Blood Pressure Reduction Criteria

Patient is experiencing symptoms that, in the investigator's opinion, could be associated with a reduction in blood pressure and is also experiencing at least one of the following, confirmed by retest within 15 minutes:
Seated systolic blood pressure <100 mmHg or seated diastolic blood pressure <60 mmHg
Decrease of seated systolic blood pressure of ≥20 mmHg from the previous visit or decrease of seated diastolic blood pressure of ≥10 mmHg from the previous visit
Orthostatic hypotension
Orthostatic tachycardia

TABLE 27
Summary of Prespecified Asymptomatic or Symptomatic
Blood Pressure Reduction Criteria and Dose Reductions

| | PPF Cohort | | |
|---|---|---|---|
| | | 30-mg | 60-mg |
| | Placebo | Compound A | Compound A |
| Patients, no. (%) | (n = 41) | (n = 40) | (n = 42) |
| Asymptomatic blood | 6 (14.6) | 9 (22.5) | 13 (31.0) |
| pressure reduction* | | | |
| Orthostatic | 6 (14.6) | 6 (15.0) | 11 (26.2) |
| hypotension^{†} | | | |
| Symptomatic blood | 2 (4.9) | 2 (5.0) | 6 (14.3) |
| pressure reduction* | | | |
| Orthostatic | 1 (2.4) | 1 (2.5) | 4 (9.5) |
| hypotension^{†} | | | |
| Dose reductions | 1 (2.4) | 6 (15.0) | 5 (11.9) |

*The prespecified criteria that define asymptomatic versus symptomatic blood pressure reduction are shown in the Supplementary Methods.
^{†}Orthostatic hypotension was defined as a drop in systolic blood pressure of ≥20 mmHg or diastolic blood pressure of ≥10 mmHg with an assumption of an upright posture from either supine or seated to upright position.
IPF, idiopathic pulmonary fibrosis; PPF, progressive pulmonary fibrosis.

Compound A was well-tolerated, demonstrated no unexpected safety findings, and had an overall profile similar to that seen in the IPF cohort. Other than low blood pressure, the adverse event profile was generally in favor of the 60 mg group versus placebo.

There was a dose dependent day-1 blood pressure reduction in the PPF cohort, similar in magnitude to what was seen in the IPF cohort. Orthostatic hypotension (asymptomatic and symptomatic) was higher in the 60 mg group than for other groups. There were 2 cases of syncope and 1 case of presyncope, but all were in the placebo group.

It is to be appreciated that the Detailed Description section, and not the Summary and Abstract sections, is intended to be used to interpret the claims. The Summary and Abstract sections may set forth one or more but not all exemplary aspects of the present disclosure as contemplated by the inventor(s), and thus, are not intended to limit the present disclosure and the appended claims in any way.

The present disclosure has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific aspects will so fully reveal the general nature of the disclosure that others can, by applying knowledge within the skill of the art, readily modify and/or adapt for various applications such specific aspects, without undue experimentation, without departing from the general concept of the present disclosure. Therefore, such adaptations and modifications are intended to be within the meaning and range of equivalents of the disclosed aspects, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present disclosure should not be limited by any of the above-described exemplary aspects, but should be defined only in accordance with the following claims and their equivalents.

1. A method of treating interstitial lung disease, the method comprising administering to a subject in need thereof about 120 mg/day of Compound A: 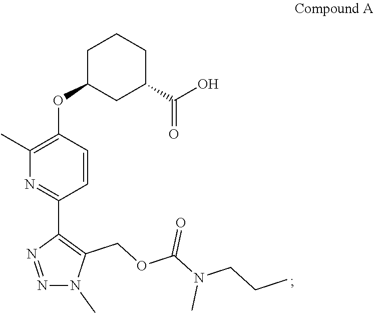 [SMILES: CCCN(C)C(=O)OCc1c(-c2ccc(O[C@H]3CCC[C@H](C(=O)O)C3)c(C)n2)nnn1C]
or an equivalent amount of a pharmaceutically acceptable salt thereof.

2. The method of claim 1, wherein Compound A, or the pharmaceutically acceptable salt thereof, is administered once daily.

3. The method of claim 1, wherein Compound A, or the pharmaceutically acceptable salt thereof, is administered twice daily.

4. The method of claim 3, wherein about 60 mg of Compound A, or an equivalent amount of a pharmaceutically acceptable salt thereof, is administered twice daily.

5. The method of claim 1, wherein Compound A, or the pharmaceutically acceptable salt thereof, is administered orally.

6. The method of claim 5, wherein Compound A, or the pharmaceutically acceptable salt thereof, is administered as a tablet.

7. The method of claim 1, wherein the subject is concomitantly being treated with one or more therapies for interstitial lung disease.

8. The method of claim 7 wherein the one or more therapies is pirfenidone.

9. The method of claim 7, wherein the one or more therapies is ninedanib.

10. The method of claim 1, wherein Compound A, or the pharmaceutically acceptable salt thereof, is administered with food.

11. The method of claim 1, wherein Compound A, or the pharmaceutically acceptable salt thereof, is administered without food.

12. The method of claim 1, wherein the interstitial lung disease is idiopathic pulmonary fibrosis (IPF).

13. The method of claim 1, wherein the interstitial lung disease is progressive pulmonary fibrosis (PPF).

14. The method of claim 1, wherein Compound A comprises the crystal form characterized by at least one of the following:
a) single crystal structure having unit cell parameters substantially equal to

| Crystal system, space group | Triclinic, P1 | |
|---|---|---|
| Unit cell dimensions | a = 6.53 ± | alpha = |
| | 0.10 \acute{Å} | 92.8 ± 1.0° |
| | b = 13.06 ± | beta = |
| | 0.10 \acute{Å} | 95.5 ± 1.0° |
| | c = 14.04 ± | gamma = |
| | 0.10 \acute{Å} | 93.0 ± 1.0° |
| | |
|---|---|
| Volume | 1189(20) \acute{Å}³ |
| Density (calculated) | 1.239 g/cm³ |
| Temperature | room temperature |

wherein measurement of the single crystal structure is at room temperature;
b) a powder x-ray diffraction pattern substantially the same as shown in FIG. 1;
c) a powder x-ray diffraction pattern comprising 2 or more peaks at 2θ values selected from 6.4±0.2, 6.8±0.2, 9.6±0.2, 13.6±0.2, 15.7±0.2, 18.2±0.2, 19.9±0.2, 21.6±0.2, 24.8±0.2 and 26.8±0.2 (obtained at room temperature and CuKαλ=1.5418 Å);
d) a powder x-ray diffraction pattern comprising 3 or more peaks at 2θ values selected from 6.4±0.2, 6.8±0.2, 9.6±0.2, 13.6±0.2, 14.1±0.2, 14.5±0.2, 14.7±0.2, 15.7±0.2, 18.2±0.2, 18.7±0.2, 19.2±0.2, 19.9±0.2, 20.5±0.2, 21.6±0.2, 22.5±0.2, 23.1±0.2, 24.1±0.2, 24.8±0.2, 25.6±0.2, 26.8±0.2, 27.1±0.2 and 27.8±0.2 (obtained at room temperature and CuKαλ=1.5418 Å);
e) a differential scanning calorimetry thermogram substantially similar to the one as shown in FIG. 2;
f) a differential scanning calorimetry thermogram with an endotherm having an onset at about 152° C.; and/or
g) a thermal gravimetric analysis thermogram substantially similar to the one as shown in FIG. 3.

15. The method of claim 1, wherein the subject experiences a smaller decline in forced vital capacity (FVC) after a treatment period compared to an untreated subject.

16. The method of claim 1, wherein the subject experiences a greater time to first disease progression event after a treatment period than an untreated subject, wherein the first disease progression event is selected from:
absolute predicted forced vital capacity (ppFVC) decline of ≥10% from baseline;
acute exacerbation of lung fibrosis;
lung fibrosis-related hospitalization;
and
all-cause mortality.

17. The method of claim 1, wherein the subject experiences a smaller increase in cough domain score as measured by the Living with Pulmonary Fibrosis (L-PF) questionnaire over a treatment period than an untreated subject.

18. The method of claim 1, wherein the subject experiences a smaller increase in dyspnea score as measured by the Living with Pulmonary Fibrosis (L-PF) questionnaire over a treatment period than an untreated subject.

19.-22. (canceled)
